SEQUENCE LISTING (28 sequences)

SEQ ID NO: 1 - protein, 299 aa, Homo sapiens
MGTKAQVERKLLCLFILAILLCSLALGSVTVHSSEPEVRIPENNPVKLSCAYSGFSSPRVEWKFDQGDTTRLVCYNNKITASYEDRVTFLPTGITFKSVTREDTGTYTCMVSEEGGNSYGEVKVKLIVLVPPSKPTVNIPSSATIGNRAVLTCSEQDGSPPSEYTWFKDGIVMPTNPKSTRAFSNSSYVLNPTTGELVFDPLSASDTGEYSCEARNGYGTPMTSNAVRMEAVERNVGVIVAAVLVTLILLGILVFGIWFAYSRGHFDRTKKGTSSKKVIYSQPSARSEGEFKQTSSFLV

SEQ ID NO: 2 - protein, 298 aa, Homo sapiens
MARRSRHRFLLLLLRYLVVALGYHKAYGFSAPKDQQVVTAVEYQEAILACKTPKKTVSSRLEWKKLGRSVSFVYYQQTLQGDFKNRAEMIDFNIRIKNVTRSDAGKYRCEVSAPSEQGQNLEEDTVTLEVLVAPAVPSCEVPSSALSGTVVELRCQDKEGNPAPEYTWFKDGIRLLENPRLGSQSTNSSYTMNTKTGTLQFNTVSKLDTGEYSCEARNSVGYRRCPGKRMQVDDLNISGIIAAVVVVALVISVCGLGVCYAQRKGYFSKETSFQKSNSSSKATTMSENDFKHTKSFII

SEQ ID NO: 3 - protein, 310 aa, Homo sapiens
MALRRPPRLRLCARLPDFFLLLLFRGCLIGAVNLKSSNRTPVVQERESVELSCIITDSQTSDPRIEWKKIQDEQTTYVFFDNKIQGDLAGRAEILGKTSLKIWNVTRRDSALYRCEVVARNDRKEIDEIVIELTVWVKPVTPVCRVPKAVPVGKMATLHCQESEGHPRPHYSWYRNDVPLPTDSRANPRFRNSSFHLNSETGTLVFTAVHKDDSGQYYCIASNDAGSARCEEQEMEVYDLNIGGIIGGVLVVLAVLALITLGICCAYRRGYFINNKQDGESYKNPGKPDGVNYIRTDEEGDFRHKSSFVI

SEQ ID NO: 4 - DNA, 900 bp, Homo sapiens
atggggacaaaggcgcaagtcgagaggaaactgttgtgcctcttcatattggcgatcctgttgtgctccctggcattgggcagtgttacagtgcactcttctgaacctgaagtcagaattcctgagaataatcctgtgaagttgtcctgtgcctactcgggcttttcttctccccgtgtggagtggaagtttgaccaaggagacaccaccagactcgtttgctataataacaagatcacagcttcctatgaggaccgggtgaccttcttgccaactggtatcaccttcaagtccgtgacacgggaagacactgggacatacacttgtatggtctctgaggaaggcggcaacagctatggggaggtcaaggtcaagctcatcgtgcttgtgcctccatccaagcctacagttaacatcccctcctctgccaccattgggaaccgggcagtgctgacatgctcagaacaagatggttccccaccttctgaatacacctggttcaaagatgggatagtgatgcctacgaatcccaaaagcacccgtgccttcagcaactcttcctatgtcctgaatcccacaacaggagagctggtctttgatcccctgtcagcctctgatactggagaatacagctgtgaggcacggaatgggtatgggacacccatgacttcaaatgctgtgcgcatggaagctgtggagcggaatgtgggggtcatcgtggcagccgtccttgtaaccctgattctcctgggaatcttggtttttggcatctggtttgcctatagccgaggccactttgacagaacaaagaaagggacttcgagtaagaaggtgatttacagccagcctagtgcccgaagtgaaggagaattcaaacagacctcgtcattcctggtgtga

SEQ ID NO: 5 - DNA, 897 bp, Homo sapiens
atggcgaggaggagccgccaccgcttcctcctgctgctgctgcgctacctggtggtcgccctgggctatcataaggcctatgggttttctgccccaaaagaccaacaggtagtcacagcagtagagtaccaagaggctattttagcctgcaaaaccccaaagaagactgtttcctccagattagagtggaagaaactgggtcggagtgtctcctttgtctactatcaacagactcttcaaggtgattttaaaaatcgagctgagatgatagatttcaatatccggatcaaaaatgtgacaagaagtgatgcggggaaatatcgttgtgaagttagtgccccatctgagcaaggccaaaacctggaagaggatacagtcactctggaagtattagtggctccagcagttccatcatgtgaagtaccctcttctgctctgagtggaactgtggtagagctacgatgtcaagacaaagaagggaatccagctcctgaatacacatggtttaaggatggcatccgtttgctagaaaatcccagacttggctcccaaagcaccaacagctcatacacaatgaatacaaaaactggaactctgcaatttaatactgtttccaaactggacactggagaatattcctgtgaagcccgcaattctgttggatatcgcaggtgtcctgggaaacgaatgcaagtagatgatctcaacataagtggcatcatagcagccgtagtagttgtggccttagtgatttccgtttgtggccttggtgtatgctatgctcagaggaaaggctacttttcaaaagaaacctccttccagaagagtaattcttcatctaaagccacgacaatgagtgaaaatgatttcaagcacacaaaatcctttataatttaa

SEQ ID NO: 6 - DNA, 933 bp, Homo sapiens
atggcgctgaggcggccaccgcgactccggctctgcgctcggctgcctgacttcttcctgctgctgcttttcaggggctgcctgataggggctgtaaatctcaaatccagcaatcgaaccccagtggtacaggaatttgaaagtgtggaactgtcttgcatcattacggattcgcagacaagtgaccccaggatcgagtggaagaaaattcaagatgaacaaaccacatatgtgttttttgacaacaaaattcagggagacttggcgggtcgtgcagaaatactggggaagacatccctgaagatctggaatgtgacacggagagactcagccctttatcgctgtgaggtcgttgctcgaaatgaccgcaaggaaattgatgagattgtgatcgagttaactgtgcaagtgaagccagtgacccctgtctgtagagtgccgaaggctgtaccagtaggcaagatggcaacactgcactgccaggagagtgagggccacccccggcctcactacagctggtatcgcaatgatgtaccactgcccacggattccagagccaatcccagatttcgcaattcttcttcccacttaaactctgaaacaggcactttggtgttcactgctgttcacaaggacgactctgggcagtactactgcattgcttccaatgacgcaggctcagccaggtgtgaggagcaggagatggaagtctatgacctgaacattggcggaattattgggggggttctggttgtccttgctgtactggccctgatcacgttgggcatctgctgtgcatacagacgtggctacttcatcaacaataaacaggatggagaaagttacaagaacccagggaaaccagatggagttaactacatccgcactgacgaggagggcgacttcagacacaagtcatcgtttgtgatctga

SEQ ID NO: 7 - protein, 235 aa, Homo sapiens
MGTKAQVERKLLCLFILAILLCSLALGSVTVHSSEPEVRIPENNPVKLSCAYSGFSSPRVEWKFDQGDTTRLVCYNNKITASYEDRVTFLPTGITFKSVTREDTGTYTCMVSEEGGNSYGEVKVKLIVLVPPSKPTVNIPSSATIGNRAVLTCSEQDGSPPSEYTWFKDGIVMPTNPKSTRAFSNSSYVLNPTTGELVFDPLSASDTGEYSCEARNGYGTPMTSNAVRMEAVERN

SEQ ID NO: 8 - protein, 208 aa, Homo sapiens
SVTVHSSEPEVRIPENNPVKLSCAYSGFSSPRVEWKFDQGDTTRLVCYNNKITASYEDRVTFLPTGITFKSVTREDTGTYTCMVSEEGGNSYGEVKVKLIVLVPPSKPTVNIPSSATIGNRAVLTCSEQDGSPPSEYTWFKDGIVMPTNPKSTRAFSNSSYVLNPTTGELVFDPLSASDTGEYSCEARNGYGTPMTSNAVRMEAVERN

SEQ ID NO: 9 - protein, 223 aa, Homo sapiens
MARRSRHRFLLLLLRYLVVALGYHKAYGFSAPKDQQVVTAVEYQEAILACKTPKKTVSSRLEWKKLGRSVSFVYYQQTLQGDFKNRAEMIDFNIRIKNVTRSDAGKYRCEVSAPSEQGQNLEEDTVTLEVLVAPAVPSCEVPSSALSGTVVELRCQDKEGNPAPEYTWFKDGIRLLENPRLGSQSTNSSYTMNTKTGTLQFNTVSKLDTGEYSCEARNSVGYR

SEQ ID NO: 10 - protein, 236 aa, Homo sapiens
MARRSRHRFLLLLLRYLVVALGYHKAYGFSAPKDQQVVTAVEYQEAILACKTPKKTVSSRLEWKKLGRSVSFVYYQQTLQGDFKNRAEMIDFNIRIKNVTRSDAGKYRCEVSAPSEQGQNLEEDTVTLEVLVAPAVPSCEVPSSALSGTVVELRCQDKEGNPAPEYTWFKDGIRLLENPRLGSQSTNSSYTMNTKTGTLQFNTVSKLDTGEYSCEARNSVGYRRCPGKRMQVDDLN

SEQ ID NO: 11 - protein, 196 aa, Homo sapiens
FSAPKDQQVVTAVEYQEAILACKTPKKTVSSRLEWKKLGRSVSFVYYQQTLQGDFKNRAEMIDFNIRIKNVTRSDAGKYRCEVSAPSEQGQNLEEDTVTLEVLVAPAVPSCEVPSSALSGTVVELRCQDKEGNPAPEYTWFKDGIRLLENPRLGSQSTNSSYTMNTKTGTLQFNTVSKLDTGEYSCEARNSVGYRR

SEQ ID NO: 12 - protein, 208 aa, Homo sapiens
FSAPKDQQVVTAVEYQEAILACKTPKKTVSSRLEWKKLGRSVSFVYYQQTLQGDFKNRAEMIDFNIRIKNVTRSDAGKYRCEVSAPSEQGQNLEEDTVTLEVLVAPAVPSCEVPSSALSGTVVELRCQDKEGNPAPEYTWFKDGIRLLENPRLGSQSTNSSYTMNTKTGTLQFNTVSKLDTGEYSCEARNSVGYRRCPGKRMQVDDLN

SEQ ID NO: 13 - protein, 238 aa, Homo sapiens
MALRRPPRLRLCARLPDFFLLLLFRGCLIGAVNLKSSNRTPVVQEFESVELSCIITDSQTSDPRIEWKKIQDEQTTYVFFDNKIQGDLAGRAEILGKTSLKIWNTRRDSALRCEVVARNDRKEIDEIVIELTVQVKPVTPVCRVPKAVPVGKMATLHCQESEGHPRPHYSWYRNDVPLPTDSRANPRFRNSSSHLNSETGTLVFTAVHKDDSGQYYCIASNDAGSARCEEQEMEVYDL

SEQ ID NO: 14 - protein, 210 aa, Homo sapiens
AVNLKSSNRTPVVQEFESVELSCIITDSQTSDPRIEWKKIQDEQTTYVFFDNKIQGDLAGRAEILGKTSLKIWNVTRRDSALYRCEVVARNDRKEIDEIVIELTVQVKPVTPVCRVPKAVPVGKMATLHCQESEGHPRPHYSWYRNDVPLPTDSRANPRFRNSSSHLNSETGTLVFTAVHKDDSGQYYCIASNDAGSARCEEQEMEVYDL

SEQ ID NO: 15 - DNA, 705 bp, Homo sapiens
atggggacaaaggcgcaagtcgagaggaaactgttgtgcctcttcatattggcgatcctgttgtgctccctggcattgggcagtgttacagtgcactcttctgaacctgaagtcagaattcctgagaataatcctgtgaagttgtcctgtgcctactcgggcttttcttctccccgtgtggagtggaagtttgaccaaggagacaccaccagactcgtttgctataataacaagatcacagcttcctatgaggaccgggtgaccttcttgccaactggtatcaccttcaagtccgtgacacgggaagacactgggacatacacttgtatggtctctgaggaaggcggcaacagctatggggaggtcaaggtcaagctcatcgtgcttgtgcctccatccaagcctacagttaacatcccctcctctgccaccattgggaaccgggcagtgctgacatgctcagaacaagatggttccccaccttctgaatacacctggttcaaagatgggatagtgatgcctacgaatcccaaaagcacccgtgccttcagcaactcttcctatgtcctgaatcccacaacaggagagctggtctttgatcccctgtcagcctctgatactggagaatacagctgtgaggcacggaatgggtatgggacacccatgacttcaaatgctgtgcgcatggaagctgtggagcggaat

SEQ ID NO: 16 - DNA, 672 bp, Homo sapiens
atggcgaggaggagccgccaccgcttcctcctgctgctgctgcgctacctggtggtcgccctgggctatcataaggcctatgggttttctgccccaaaagaccaacaggtagtcacagcagtagagtaccaagaggctattttagcctgcaaaaccccaaagaagactgtttcctccagattagagtggaagaaactgggtcggagtgtctcctttgtctactatcaacagactcttcaaggtgattttaaaaatcgagctgagatgatagatttcaatatccggatcaaaaatgtgacaagaagtgatgcggggaaatatcgttgtgaagttagtgccccatctgagcaaggccaaaacctggaagaggatacagtcactctggaagtattagtggctccagcagttccatcatgtgaagtaccctcttctgctctgagtggaactgtggtagagctacgatgtcaagacaaagaagggaatccagctcctgaatacacatggtttaaggatggcatccgtttgctagaaaatcccagacttggctcccaaagcaccaacagctcatacacaatgaatacaaaaactggaactctgcaatttaatactgtttccaaactggacactggagaatattcctgtgaagcccgcaattctgttggatatcgcagg

SEQ ID NO: 17 - DNA, 708 bp, Homo sapiens
atggcgaggaggagccgccaccgcttcctcctgctgctgctgcgctacctggtggtcgccctgggctatcataaggcctatgggttttctgccccaaaagaccaacaggtagtcacagcagtagagtaccaagaggctattttagcctgcaaaaccccaaagaagactgtttcctccagattagagtggaagaaactgggtcggagtgtctcctttgtctactatcaacagactcttcaaggtgattttaaaaatcgagctgagatgatagatttcaatatccggatcaaaaatgtgacaagaagtgatgcggggaaatatcgttgtgaagttagtgccccatctgagcaaggccaaaacctggaagaggatacagtcactctggaagtattagtggctccagcagttccatcatgtgaagtaccctcttctgctctgagtggaactgtggtagagctacgatgtcaagacaaagaagggaatccagctcctgaatacacatggtttaaggatggcatccgtttgctagaaaatcccagacttggctcccaaagcaccaacagctcatacacaatgaatacaaaaactggaactctgcaatttaatactgtttccaaactggacactggagaatattcctgtgaagcccgcaattctgttggatatcgcaggtgtcctgggaaacgaatgcaagtagatgatctcaac

SEQ ID NO: 18 - DNA, 720 bp, Homo sapiens
atggcgctgaggcggccaccgcgactccggctctgcgctcggctgcctgacttcttcctgctgctgcttttcaggggctgcctgataggggctgtaaatctcaaatccagcaatcgaaccccagtggtacaggaatttgaaagtgtggaactgtcttgcatcattacggattcgcagacaagtgaccccaggatcgagtggaagaaaattcaagatgaacaaaccacatatgtgttttttgacaacaaaattcagggagacttggcgggtcgtgcagaaatactggggaagacatccctgaagatctggaatgtgacacggagagactcagccctttatcgctgtgaggtcgttgctcgaaatgaccgcaaggaaattgatgagattgtgatcgagttaactgtgcaagtgaagccagtgacccctgtctgtagagtgccgaaggctgtaccagtaggcaagatggcaacactgcactgccaggagagtgagggccacccccggcctcactacagctggtatcgcaatgatgtaccactgcccacggattccagagccaatcccagatttcgcaattcttcttcccacttaaactctgaaacaggcactttggtgttcactgctgttcacaaggacgactctgggcagtactactgcattgcttccaatgacgcaggctcagccaggtgtgaggagcaggagatggaagtctatgacctg

SEQ ID NO: 19 - DNA, 624 bp, Homo sapiens
agtgttacagtgcactcttctgaacctgaagtcagaattcctgagaataatcctgtgaagttgtcctgtgcctactcgggcttttcttctccccgtgtggagtggaagtttgaccaaggagacaccaccagactcgtttgctataataacaagatcacagcttcctatgaggaccgggtgaccttcttgccaactggtatcaccttcaagtccgtgacacgggaagacactgggacatacacttgtatggtctctgaggaaggcggcaacagctatggggaggtcaaggtcaagctcatcgtgcttgtgcctccatccaagcctacagttaacatcccctcctctgccaccattgggaaccgggcagtgctgacatgctcagaacaagatggttccccaccttctgaatacacctggttcaaagatgggatagtgatgcctacgaatcccaaaagcacccgtgccttcagcaactcttcctatgtcctgaatcccacaacaggagagctggtctttgatcccctgtcagcctctgatactggagaatacagctgtgaggcacggaatgggtatgggacacccatgacttcaaatgctgtgcgcatggaagctgtggagcggaat

SEQ ID NO: 20 - DNA, 587 bp, Homo sapiens
ttttctgccccaaaagaccaacaggtagtcacagcagtagagtaccaagaggctattttagcctgcaaaaccccaaagaagactgtttcctccagattagagtggaagaaactgggtcggagtgtctcctttgtctactatcaacagactcttcaaggtgattttaaaaatcgagctgagatgatagatttcaatatccggatcaaaaatgtgacaagaagtgatgcggggaaatatcgttgtgaagttagtgccccatctgagcaaggccaaaacctggaagaggatacagtcactctggaagtattagtggctccagcagttccatcatgtgaagtaccctcttctgctctgagtggaactgtggtagagctacgatgtcaagacaaagaagggaatccagctcctgaatacacatggtttaaggatggcatccgtttgctagaaaatcccagacttggctcccaaagcaccaacagctcatacacaatgaatacaaaaactggaactctgcaatttaatactgtttccaaactggacactgggaatattcctgtgaagcccgcaattctgttggatatcgcagg

SEQ ID NO: 21 - DNA, 623 bp, Homo sapiens
ttttctgccccaaaagaccaacaggtagtcacagcagtagagtaccaagaggctattttagcctgcaaaaccccaaagaagactgtttcctccagattagagtggaagaaactgggtcggagtgtctcctttgtctactatcaacagactcttcaaggtgattttaaaaatcgagctgagatgatagatttcaatatccggatcaaaaatgtgacaagaagtgatgcggggaaatatcgttgtgaagttagtgccccatctgagcaaggccaaaacctggaagaggatacagtcactctggaagtattagtggctccagcagttccatcatgtgaagtaccctcttctgctctgagtggaactgtggtagagctacgatgtcaagacaaagaagggaatccagctcctgaatacacatggtttaaggatggcatccgtttgctagaaaatcccagacttggctcccaaagcaccaacagctcatacacaatgaatacaaaaactggaactctgcaatttaatactgtttccaaactggacactgggaatattcctgtgaagcccgcaattctgttggatatcgcaggtgtcctgggaaacgaatgcaagtagatgatctcaac

SEQ ID NO: 22 - DNA, 630 bp, Homo sapiens
gctgtaaatctcaaatccagcaatcgaaccccagtggtacaggaatttgaaagtgtggaactgtcttgcatcattacggattcgcagacaagtgaccccaggatcgagtggaagaaaattcaagatgaacaaaccacatatgtgttttttgacaacaaaattcagggagacttggcgggtcgtgcagaaatactggggaagacatccctgaagatctggaatgtgacacggagagactcagccctttatcgctgtgaggtcgttgctcgaaatgaccgcaaggaaattgatgagattgtgatcgagttaactgtgcaagtgaagccagtgacccctgtctgtagagtgccgaaggctgtaccagtaggcaagatggcaacactgcactgccaggagagtgagggccacccccggcctcactacagctggtatcgcaatgatgtaccactgcccacggattccagagccaatcccagatttcgcaattcttcttcccacttaaactctgaaacaggcactttggtgttcactgctgttcacaaggacgactctgggcagtactactgcattgcttccaatgacgcaggctcagccaggtgtgaggagcaggagatggaagtctatgacctg

SEQ ID NO: 23 - DNA, 36 bp, Artificial Sequence
  Synthetic Construct
ttactaggctggcgcgccaccatggcgaggaggagc

SEQ ID NO: 24 - DNA, 35 bp, Artificial Sequence
  Synthetic Construct
aggaaggaatgcgcaaattataaaggattttgtgt

SEQ ID NO: 25 - DNA, 37 bp, Artificial Sequence
  Synthetic Construct
gatcggcgcgccagccaccatggcgctgaggcggcca

SEQ ID NO: 26 - DNA, 33 bp, Artificial Sequence
  Synthetic Construct
cgccggtttaaacgccaggtcatagacttccat

SEQ ID NO: 27 - DNA, 18 bp, Artificial Sequence
  Synthetic Construct
tgcaaagcttggcgcgcc

SEQ ID NO: 28 - DNA, 25 bp, Artificial Sequence
  Synthetic Construct
cttgtcgtcgtcatccttgtagtcg

(19) United States
(12) Patent Application Publication
(10) Pub. No.: US 2005/0159587 A1
(43) Pub. Date: Jul. 21, 2005

(54) Extracellular junctional adhesion molecules

(76) Inventors: Josef Georg Heuer, Indianapolis, IN (US); Rosamund Carol Smith, Greenfield, IN (US); Eric Wen Su, Carmel, IN (US)

Correspondence Address: ELI LILLY AND COMPANY;PATENT DIVISION, P.O. BOX 6288, INDIANAPOLIS, IN, 46206-6288, US

(21) Appl. No.: 10/481,090

(22) PCT Filed: Jul. 5, 2002
(86) PCT No.: PCT/US02/19800

Related U.S. Application Data
(60) Provisional application No. 60/305,752, filed on Jul. 16, 2001.
(60) Provisional application No. 60/354,345, filed on Feb. 5, 2002.

(51) Int. Cl. C07K 14/705; C07K 16/28; C12P 21/02; A61K 39/395

(52) U.S. Cl. 530/350; 424/143.1; 435/69.1; 435/320.1; 435/325; 530/388.22; 536/23.5

(57) ABSTRACT

This invention provides human extracellular junctional adhesion molecules (huJAM) and polynucleotides which identify and encode huJAM. The invention further provides methods using the molecules of the invention for treating, cancer and inflammatory, immune system, and cardiovascular disorders.

### FIELD OF THE INVENTION

This invention relates to human extracellular junctional adhesion molecules (JAM) and polynucleotides which identify and encode human extracellular JAM. The invention further provides compositions and methods using the proteins and polynucleotides of the invention for treating cancer, cardiovascular disorders, and immune system disorders such as autoimmune diseases and inflammatory disorders.

### BACKGROUND OF THE INVENTION

Junctional adhesion molecules (JAM) are members of the immunoglobulin superfamily (IgSf). JAM have two extracellular IgSf domains, a transmembrane segment, and a short cytoplasmic segment. Currently, three distinct JAM proteins, JAM1, JAM2, and JAM3, have been identified in both murine and human sources. JAM are localized to the intercellular boundaries of endothelial and epithelial cells although the tissue distribution pattern for each is distinct (Aurrand-Lions, M. et al. Curr. Top. Microbiol. Immunol. 251: 91-98, 2000).

Endothelial cells lining blood vessels form a blood-tissue barrier and such cells are attached to each other by at least two types of complex, junctional structures, adherens junctions (AJ) and tight junctions (TJ), to form a continuous layer of cells. JAM1, JAM2, and JAM3 are concentrated at the sites of cell-cell junctions for both endothelial and epithelial cells and facilitate cell-cell contact through homotypic and/or heterotypic interactions. JAM are also functionally implicated in cell trafficking and cell-fate determination (Malergue, F. et al. Mol. Immunol. 35: 1111-1119, 1998).

Leukocytes, it is commonly believed, leave the blood by first adhering to endothelial cells and then migrating through the interendothelial junctions. In doing so they cause disruption of the junctional structures. The process by which leukocytes traverse these junctions is not completely understood, particularly on a molecular level. However, it has been proposed that JAM play a structural role in the control of leukocyte migration across epithelium or endothelium to sites of inflammation. JAM1, localized to TJ, is involved in myeloid cell and neutrophil transmigration (Padura, I., et al. J. Cell Biol. 142: 117-127, 1998) while JAM2 may have a role in controlling leukocyte recirculation at secondary lymphoid organs such as lymph nodes and tonsils (Aurrand-Lions, M. et al. Curr. Top. Microbiol. Immunol. 251: 91-98, 2000). Much remains to be learned about the role of JAM in leukocyte infiltration and in the inflammation process.

Human JAM (huJAM) is reported to be expressed in circulating immune cells at high levels, both at the mRNA and at the protein level (Williams, L., et al., Mol. Immunology 36: 1175-1188, 1999). It is contemplated that a JAM polypeptide on the endothelial cell interacts with a JAM polypeptide on the immune cell thereby inducing transmembrane signaling and the passage of immune cells through the interendothelial junction.

Numerous publications and databases have reported the full-length, membrane-bound sequence of murine (mu) and human (hu) JAM1, JAM2, and JAM3 polypeptides as well as the polynucleotide sequences encoding the JAM polypeptides (e.g., International patent publications: WO9842739, WO9840483, WO9927098, WO9914241, WO0073452, WO0029583, WO0061623, WO0053758, WO0053749, WO0056754, WO0053758, WO0107459). While WO0053758 (page 6) makes mention of a transmembrane-deleted human JAM3, it does not identify the location or sequence of the transmembrane sequence nor does it identify a function of such a molecule that is distinct from that of the molecule containing the transmembrane domain.

It is well known that the regulated and coordinated expression of adhesion molecules is required for normal vascular function. During inflammation, the cell-cell interactions of the epithelial cell layer are disrupted, resulting in a leaky epithelial barrier, which in turn can lead to various inflammatory and infective disorders. Changes in the adhesion properties of vascular endothelial cells are also observed during tumor growth, wound healing, and angiogenesis. There is great clinical potential and need for extracellular junction adhesion molecules which can function as agonists and/or antagonists and thereby prevent leukocyte transmigration across adherens junctions or tight junctions and be useful for the diagnosis, prevention and treatment of cancer, cardiovascular disorders, and immune system disorders.

### SUMMARY OF THE INVENTION

The present invention addresses the need for human JAM (huJAM) agonists and/or antagonists by providing extracellular huJAM polypeptides and related compositions and methods.

The present invention embodies extracellular huJAM polypeptides (LP121A (SEQ ID NO:11), LP121B (SEQ ID NO:12), LP121C (SEQ ID NO:8), LP10034 (SEQ ID NO:14)) and their use in treating cancer, cardiovascular disorders, and immune system disorders such as autoimmune diseases and inflammatory disorders.

Amino acid residues 1 to about 30 of the full-length huJAM proteins (SEQ ID NOS: 1, 2, and 3) are a signal peptide that is removed by a signal peptidase enzyme during maturation of the huJAM protein. While extracellular huJAM polypeptides LP121A, LP121B, LP121C and LP10034 can be encoded by a nucleic acid that encodes the signal peptide (SEQ ID Nos: 15, 16, 17, and 18), this signal peptide is cleaved off and is not present in the mature, extracellular form of the protein. Alternatively, the extracellular huJAM polypeptides LP121A, LP121B, LP121C and LP10034 can be encoded by a nucleic acid that lacks sequence encoding the signal peptide (SEQ ID Nos: 19, 20, 21, and 22). Also lacking in LP121A, LP121B, LP121C and LP10034 polypeptides, regardless of whether or not the signal peptide is present, are the transmembrane domain and cytoplasmic domain that are present in full-length, membrane-bound huJAM (see FIG. 1 for the domain boundaries and Table 2 for nomenclature summary).

The invention embodies multiple forms of isolated and purified extracellular huJAM polypeptides e.g., extracellular huJAM2 also referred to herein as LP121A (SEQ ID NO:11) and LP121B (SEQ ID NO:12); extracellular huJAM1 also referred to herein as LP121C (SEQ ID NO:8); and extracellular huJAM3 also referred to herein as LP10034 (SEQ ID:14). The invention further embodies extracellular huJAM polypeptide variants with an amino acid sequence variation of SEQ ID NOS: 8, 11, 12, or 14 as further described herein. It is contemplated that the extracellular huJAM3 of the invention embodies allelic variants e.g., the variant in which the amino acid at position 195 of the full length protein, (SEQ ID NO:3), or the equivalent position in the mature protein, is either a phenylalanine or a serine. The invention further contemplates LP polypeptides that have an amino acid sequence at least about 95%, even more preferably at least 96%, 97%, or 98% and most preferably at least 99% identical (i.e., amino acid sequence identity) to that shown in SEQ ID NOS: 8, 11, 12, or 14.

The invention also embodies an expression vector encoding an extracellular huJAM polypeptide, or an extracellular huJAM polypeptide variant, operably linked to a promoter sequence and a host cell transfected with such an expression vector that produces an extracellular huJAM polypeptide. Exemplary host cells include, but are not limited to, CHO cells, E. coli cells, Sf9 cells and yeast cells. The type of promoter sequence used in the expression vector will vary depending upon the host cell type.

A further aspect of the invention embodies an isolated and purified fusion polypeptide consisting essentially of a first portion and a second portion joined by a peptide bond. The first portion of the fusion polypeptide comprises (a) an extracellular huJAM with an amino acid sequence shown in SEQ ID NOS: 8, 11, 12, or 14 as the sole source of huJAM in the fusion polypeptide, (b) an extracellular huJAM variant with an amino acid sequence variation of the sequence shown in SEQ ID NOS: 8, 11, 12, or 14 as the sole source of huJAM in the fusion polypeptide or (c) a protein polypeptide that has an amino acid sequence at least about 95%, even more preferably at least 96%, 97%, or 98% and most preferably at least 99% identical (i.e., amino acid sequence identity) to that described in (a) or (b). The second portion of the fusion polypeptide consists of another polypeptide such as an affinity tag. Within one embodiment the affinity tag is an immunoglobulin Fc polypeptide. Within another embodiment the affinity tag is FLAG or His6.

The invention also provides an expression vector encoding a fusion polypeptide and a host cell transfected with such an expression vector to produce a fusion polypeptide wherein the fusion polypeptide consists essentially of a first portion and a second portion joined by a peptide bond. The first portion of the fusion polypeptide comprises (a) an extracellular huJAM with an amino acid sequence shown in SEQ ID NOS: 8, 11, 12, or 14 as the sole source of huJAM in the fusion polypeptide, (b) an extracellular huJAM variant with an amino acid sequence variation of the sequence shown in SEQ ID NOS: 8, 11, 12, or 14 as the sole source of huJAM in the fusion polypeptide or (c) a protein polypeptide with an amino acid sequence that is at least about 95%, even more preferably at least about 96%, 97%, or 98% and most preferably at least 99% identical (i.e., amino acid sequence identity) to that described in (a) or (b). The second portion of the fusion polypeptide consists of another polypeptide such as an affinity tag. Within one embodiment the affinity tag is an immunoglobulin Fc polypeptide. Within another embodiment the affinity tag is FLAG or His6. Exemplary host cells include, but are not limited to, CHO cells, E. coli cells, Sf9 cells and yeast cells.

One embodiment of the invention provides isolated nucleic acid molecules encoding a polypeptide of the present invention including mRNAs, DNAs, cDNAs, and genomic DNAs.

The present invention also provides isolated and purified polynucleotides encoding an extracellular huJAM. More specifically, such polynucleotides have the DNA sequence shown in (a) SEQ ID NOS: 15, 16, 17, and 18, (b) complements of SEQ ID NOS: 15, 16, 17, and 18, (c) isolated polynucleotides, preferably DNA, that hybridize to (a) or (b) and not to a huJAM transmembrane domain (as identified in FIGS. 1-6 herein), under stringent hybridization and wash conditions, and (d) nucleic acid molecules, preferably DNA, comprising a nucleotide sequence having at least 90%, 91%, 92%, 93%, or 94% or more preferably at least 95%, 96%, 97%, or 98% or most preferably at least 99% nucleic acid sequence identity to a nucleic acid molecule of (a), (b), or (c).

While SEQ ID NOS: 15, 16, 17, and 18 have nucleic acid sequence encoding the signal peptide, it is contemplated that polynucleotides encoding an extracellular huJAM can lack the nucleic acid sequence encoding the signal peptide and fall within the bounds of the invention. Such polynucleotides have the DNA sequence shown in (e) SEQ ID NOS: 19, 20, 21, and 22, (f) complements of SEQ ID NOS: 19, 20, 21, and 22, (g) polynucleotides, preferably DNA, that hybridize to (e) or (f) and not to a huJAM transmembrane domain under stringent hybridization and wash conditions, and (h) nucleic acid molecules, preferably DNA, comprising a nucleotide sequence having at least about 95%, 96%, 97%, or 98% or most preferably at least 99% nucleic acid sequence identity to a nucleic acid molecule of (e), (f), or (g).

Additional compositions of the invention are those comprising: (a) a purified, therapeutically effective, extracellular huJAM polypeptide with an amino acid sequence shown in SEQ ID NOS: 8, 11, 12, or 14 or variants thereof as the sole source of huJAM sequence; or a purified, therapeutically effective extracellular huJAM polypeptide with an amino acid sequence that is at least about 95%, even more preferably at least 96%, 97%, or 98% and most preferably at least 99% identical to the amino acid sequence shown in SEQ ID NOS: 8, 11, 12, or 14 as the sole source of huJAM sequence; or a purified, therapeutically effective fusion protein comprising SEQ ID NOS: 8, 11, 12, or 14 or variations thereof as the sole source of huJAM sequence and (b) a sterile binding compound or binding agent, or the binding compound or binding agent and a carrier, wherein the carrier is: an aqueous compound including water, saline, and/or buffer; and formulated for oral, rectal, nasal, topical, or parenteral administration. As used herein “as the sole source of huJAM sequence” means that there is no transmembrane domain, cytoplasmic domain, and no signal peptide sequence present that originates from huJAM. It is, however, contemplated that a composition can comprise one, two, or more different extracellular huJAM.

In other embodiments, the invention provides a method of modulating the physiology or development of a cell in vivo or in situ comprising introducing into such cell, or the environment of such cell, a therapeutically effective amount of LP121A (SEQ ID NO: 11), LP121B (SEQ ID NO: 12), LP121C (SEQ ID NO: 8), or LP10034 (SEQ ID NO: 14) or a variant thereof, or a fusion protein comprising LP121A (SEQ ID NO:11), LP121B (SEQ ID NO: 12), LP121C (SEQ ID NO: 8), or LP10034 (SEQ ID NO: 14), or a variant thereof, as the sole source of huJAM in the fusion protein.

### BRIEF DESCRIPTION OF THE FIGURES

FIG. 1 provides an alignment of full-length membrane-bound huJAM1 (SEQ ID NO: 1), huJAM2 (SEQ ID NO: 2) and huJAM3 (SEQ ID NO: 3) polypeptide sequences with consensus amino acids, putative signal sequences, transmembrane domains and cytoplasmic domains identified.

FIG. 2A provides the polynucleotide sequence encoding huJAM1 (SEQ ID NO: 4) and FIG. 2B provides the amino acid sequence of full-length huJAM1 (SEQ ID NO: 1). The signal peptide and the nucleic acid sequence encoding it are in bold, the transmembrane and cytoplasmic regions 3′ to the extracellular domain and the nucleic acid sequence encoding it are underlined.

FIG. 3A provides the polynucleotide sequence encoding huJAM2 (SEQ ID NO: 5) and FIG. 3B provides the amino acid sequence of full-length huJAM2 (SEQ ID NO:2). The signal peptide and the nucleic acid sequence encoding it are in bold, the transmembrane and cytoplasmic regions 3′ to the extracellular domain and the nucleic acid sequence encoding it are underlined.

FIG. 4A provides the polynucleotide sequence encoding huJAM3 (SEQ ID NO: 6) and FIG. 4B provides the amino acid sequence of full-length huJAM3 (SEQ ID NO: 3). The signal peptide and the nucleic acid sequence encoding it are in bold, the transmembrane and cytoplasmic regions 3′ to the extracellular domain and the nucleic acid sequence encoding it are underlined.

FIG. 5 provides the amino acid sequence of the signal peptide and extracellular domain of huJAM1 comprising amino acids 1-235 of full-length huJAM 1 (SEQ ID NO: 7).

FIG. 6 provides the amino acid sequence of the extracellular domain of huJAM1 comprising amino acids 28-235 of full-length huJAM1 (SEQ ID NO: 8).

FIG. 7A provides the amino acid sequence of the signal peptide and extracellular domain of huJAM2 comprising amino acids 1-224 of the full-length huJAM2 (SEQ ID NO: 9).

FIG. 7B provides the amino acid sequence of the signal peptide and extracellular domain of huJAM2 comprising amino acids 1-236 of the full-length huJAM2 (SEQ ID NO: 10).

FIG. 8A provides the amino acid sequence of the extracellular domain of huJAM2 comprising amino acids 29-224 of the full-length huJAM2 (SEQ ID NO: 11).

FIG. 8B provides the amino acid sequence of the extracellular domain of huJAM2 comprising amino acids 29-236 of the full-length huJAM2 (SEQ ID NO: 12).

FIG. 9 provides the amino acid sequence of the signal peptide and extracellular domain of huJAM3 comprising amino acids 1-240 of the full-length huJAM3 (SEQ ID NO: 13).

FIG. 10 provides the amino acid sequence of the extracellular domain of huJAM3 comprising amino acids 31-240 of the full-length huJAM3 (SEQ ID NO: 14).

FIG. 11 provides the nucleotide sequence encoding the signal peptide and extracellular domain of huJAM1 (amino acids 1-235 of SEQ ID NO: 1)(SEQ ID NO: 15).

FIG. 12A provides the nucleotide sequence encoding the signal peptide and extracellular domain of huJAM2 (amino acids 1-224 of SEQ ID NO: 2) (SEQ ID NO: 16).

FIG. 12B provides the nucleotide sequence encoding the signal peptide and extracellular domain of huJAM2 (amino acids 1-236 of SEQ ID NO: 2) (SEQ ID NO: 17).

FIG. 13 provides the nucleotide sequence encoding the signal peptide and extracellular domain of huJAM3 (amino acids 1-240 of SEQ ID NO: 3) (SEQ ID NO: 18).

FIG. 14 provides the nucleotide sequence encoding the extracellular domain of huJAM1 (amino acids 28-235 of SEQ ID NO: 1) (SEQ ID NO: 19).

FIG. 15A provides the nucleotide sequence encoding the extracellular domain of huJAM2 (amino acids 29-224 of SEQ ID NO: 2) (SEQ ID NO: 20).

FIG. 15B provides the nucleotide sequence encoding the extracellular domain of huJAM2 (amino acids 29-236 of SEQ ID NO: 2) (SEQ ID NO: 21).

FIG. 16 provides the nucleotide sequence encoding the extracellular domain of huJAM3 (amino acids 31-240 of SEQ ID NO: 3) (SEQ ID NO: 22).

FIG. 17 provides the percent survival of mice injected with LP121A and challenged with a lethal dose of LPS.

FIG. 18 provides murine serum TNF-α levels two hours after mice injected with LP121A are challenged with a lethal dose of LPS.

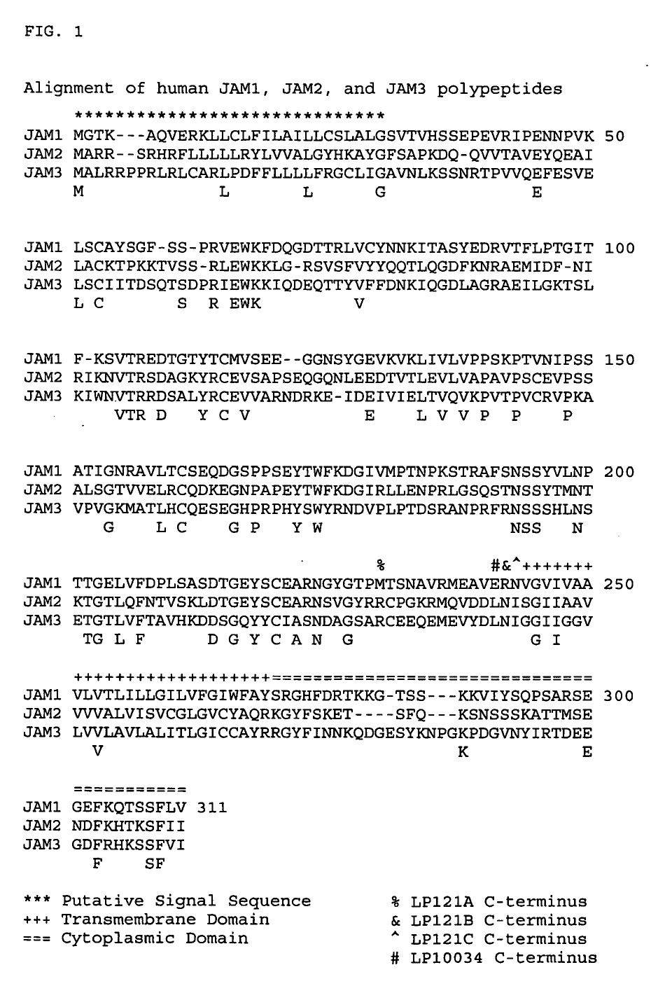

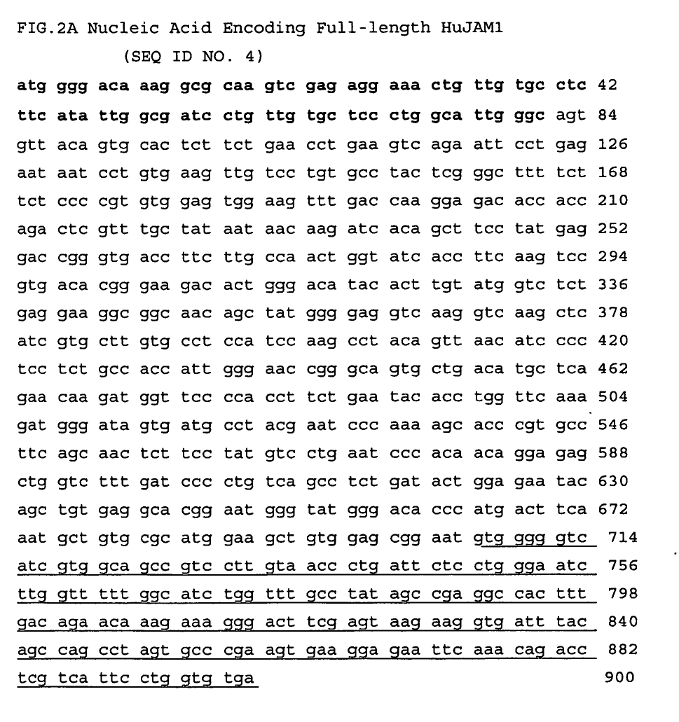

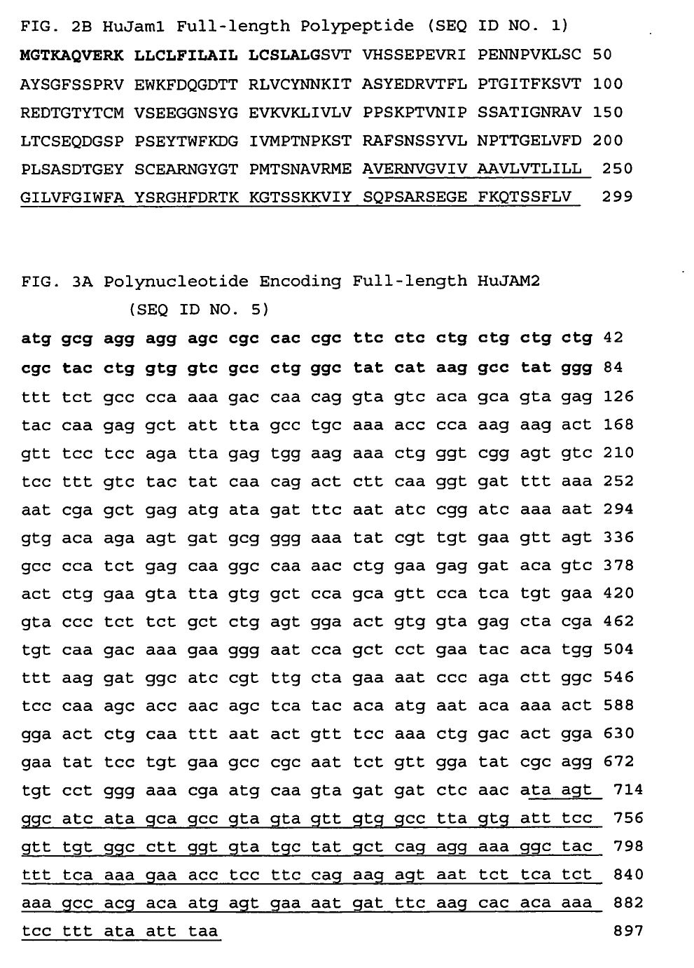

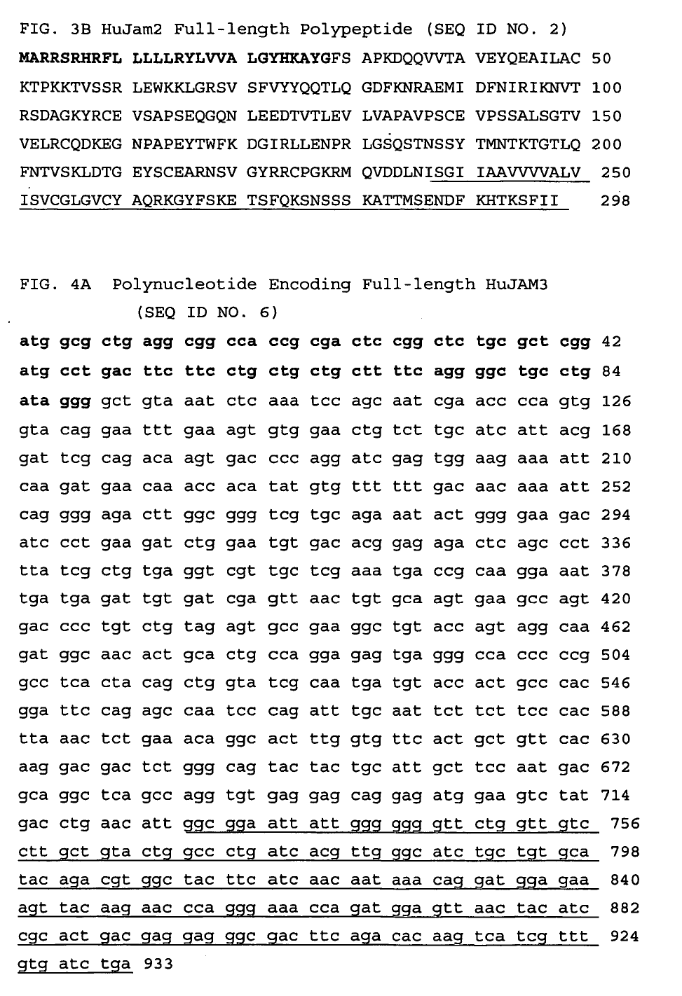

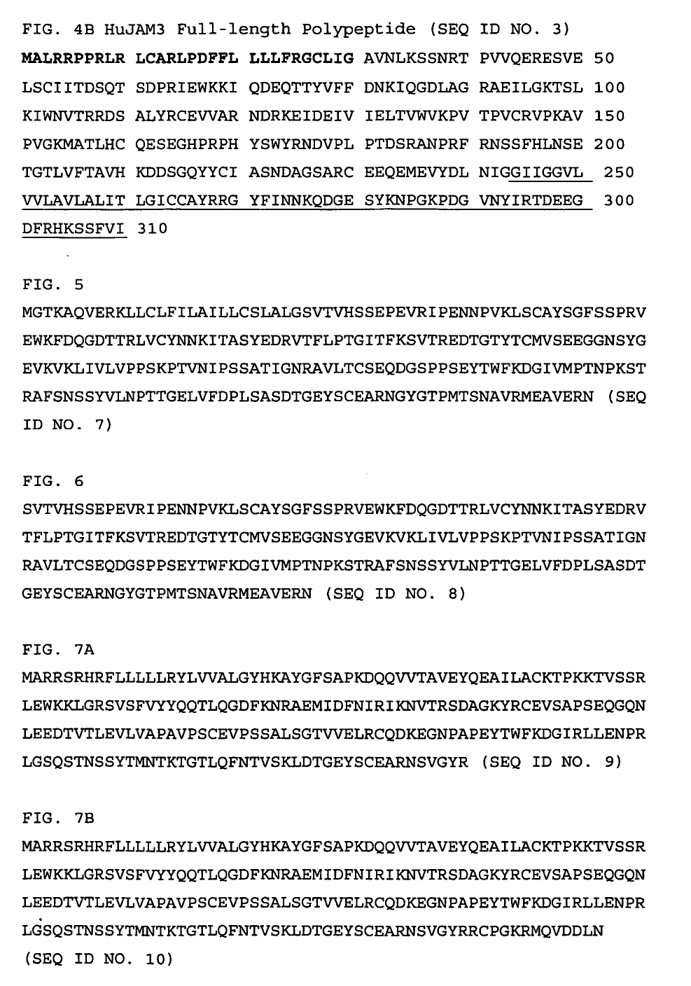

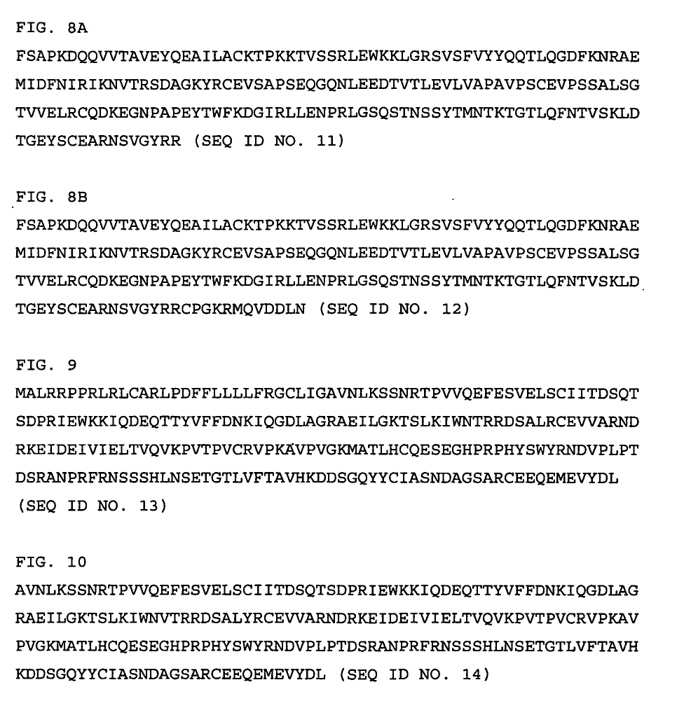

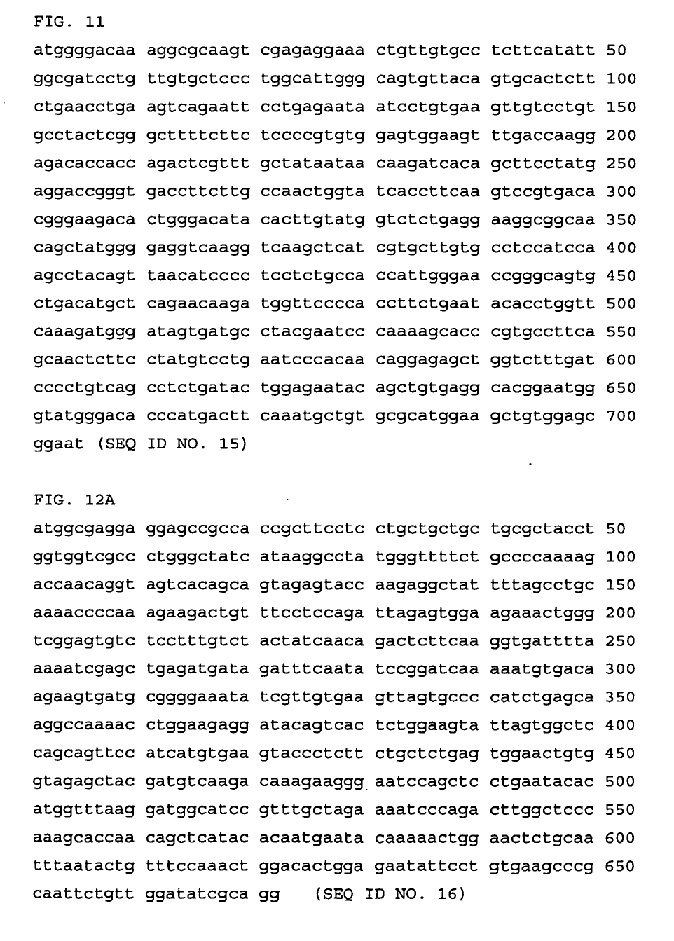

### DETAILED DESCRIPTION OF THE INVENTION

The invention is not limited to the particular embodiments described below, as variations of the particular embodiments may be made and still fall within the scope of the appended claims. The terminology used herein is for the purpose of describing particular embodiments, and is not intended to be limiting. Instead, the scope of the present invention will be established by the appended claims.

In this specification and the appended claims, the singular forms “a”, “an”, and “the” include plural reference unless the context clearly dictates otherwise. Unless defined otherwise, all technical and scientific terms used herein have the same meaning as commonly understood to one of ordinary skill in the art to which this invention belongs.

Definitions

To facilitate understanding of the invention, the following terms are defined.

The terms “LP polypeptide(s)” and “LP” as used herein refer to various polypeptides. The complete designation of LP immediately followed by a number (LP121A, LP121B, LP121C, LP10034) refers to a particular polypeptide sequence as described herein. The LP polypeptides described herein may be isolated from a variety of sources including, but not limited to, tissue culture media of mammalian cells expressing the LP polypeptide, lysed E. coli expressing the LP polypeptide, yeast, or Sf9 cells expressing the LP polypeptide, or prepared by recombinant or synthetic methods.

The term “isolated” when used in relation to a nucleic acid or protein, means the material is identified and separated from at least one contaminant with which it is ordinarily associated in its natural source. Such a nucleic acid could be part of a vector and/or such nucleic acid or protein could be part of a composition, and still be isolated in that such vector or composition is not part of its natural environment.

As used herein, the term “purified” means the result of any process that removes from a sample a contaminant from the component of interest, such as a protein or nucleic acid. The percent of a purified component is thereby increased in the sample.

As used herein, the term “in situ” is used in reference to activities, methods, functions and the like that occur in cell culture conditions while the term “in vivo” is used in reference to activities, methods, functions and the like that occur in an organism.

As used herein, the terms “complementary” or “complementarity” are used in reference to nucleic acids (i.e., a sequence of nucleotides) related by the well-known base-pairing rules that A pairs with T and C pairs with G. For example, the sequence 5′-A-G-T-3′, is complementary to the sequence 3′-T-C-A-5′. Complementarity between two single-stranded molecules can be “partial,” in which only some of the nucleic acid bases are matched according to the base pairing rules. On the other hand, there may be “complete” or “total” complementarity between the nucleic acid strands when all of the bases are matched according to base pairing rules. The degree of complementarity between nucleic acid strands has significant effects on the efficiency and strength of hybridization between nucleic acid strands as known well in the art.

As used herein, the term “hybridization” is used in reference to the pairing of complementary nucleic acid strands. Hybridization and the strength of hybridization (i.e., the strength of the association between nucleic acid strands) is impacted by many factors well known in the art including the degree of complementarity between the nucleic acids, level of stringency involved is affected by such conditions as the concentration of salts, the Tm (melting temperature) of the formed hybrid, the presence of other components (e.g., the presence or absence of polyethylene glycol), the molarity of the hybridizing strands and the G:C content of the nucleic acid strands.

As used herein, the term “stringency” is used in reference to the conditions of temperature, ionic strength, and the presence of other compounds, under which nucleic acid hybridizations are conducted. Hybridization generally depends on the ability of denatured DNA to reanneal when complementary strands are present in an environment below their melting temperature. With “high stringency” or “highly stringent” or “stringent” conditions, nucleic acid base pairing will occur only between nucleic acid fragments that have a high frequency of complementary base sequences. The art knows well that numerous equivalent conditions can be employed to comprise high stringency conditions. “Stringent conditions” or “high stringency conditions”, as defined herein, are identified by those that (1) employ low ionic strength and high temperature for washing. The higher the degree of desired homology between the two nucleic acid strands being hybridized, the higher the relative temperature that can be used. As a result, it follows that higher relative temperatures would tend to make the reactions more stringent, while lower temperatures less so. For additional details and explanation of stringency of hybridization reactions, see Ausubel et al., Current Protocols in Molecular Biology, Wiley Interscience Publishers, 1995 and supplements. Exemplary “high stringency” or “stringent” conditions include hybridization conditions of an overnight incubation of the two denatured nucleic acid strands at 42° C. in a solution comprising 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared, salmon sperm DNA, followed by wash conditions of 68° C. in the presence of about 0.2×SSC and about 0.1% sodium dodecyl sulfate (SDS), for one hour. (Reagents available from Sigma Corp., St. Louis, Mo.) SSC concentration may be varied from about 0.1× to 2.0×SSC, with SDS optionally being present at about 0.1% (w/v). Typically, blocking reagents are used to block non-specific hybridization. Such blocking reagents include, for instance, denatured salmon sperm DNA at about 100-200 μg/ml. Organic solvent, such as formamide at a concentration of about 35-50% (v/v) may also be used under particular circumstances, such as for RNA:DNA hybridizations. Useful variations on these wash conditions will be readily apparent to those of ordinary skill in the art. Hybridization, particularly under stringent conditions, may be suggestive of evolutionary similarity between the nucleic acids. Such similarity is strongly indicative of a similar role for the nucleic acid molecules and the polypeptides they encode.

The term “homology,” as used herein, refers to a degree of complementarity. There can be partial homology or complete homology (i.e., identity). A partially complementary sequence that at least partially inhibits a completely complementary sequence from hybridizing to a target nucleic acid is referred to using the functional term “substantially homologous.”

In the present invention “extracellular huJAM” refers to a form of the said human JAM polypeptide which is essentially free of the signal peptide and the transmembrane and cytoplasmic domains of the full-length huJAM polypeptide. The exact boundaries of where the signal peptide ends and the extracellular domain begins and the exact boundaries of where the extracellular domain ends and the transmembrane domain begins may vary but most likely by no more than about six amino acids at either end of the domain as identified herein. Therefore, an extracellular huJAM signal peptide/extracellular domain boundary as identified in the Examples, Figures, or specification may be shifted in either direction (upstream or downstream) by 6, 5, 4, 3, 2, 1, or 0 amino acids. Additionally, an extracellular huJAM extracellular domain/transmembrane domain boundary as identified in the Examples or specification may be shifted in either direction by 6, 5, 4, 3, 2, 1 or 0 amino acids. All such polypeptides and the nucleic acid molecules encoding them are contemplated by the present invention. For example, extracellular HuJAM1 domain is contemplated to extend from amino acids 28-235 of the full-length HuJAM1 polypeptide (SEQ ID NO:1), but it is contemplated that extracellular HuJAM1 could span from an amino-terminal amino acid chosen from between amino acids 22 through 34 ((inclusive) of the full-length HuJAM1 through a carboxy-terminal amino acid from between amino acids 229 through 241 (inclusive) of the full-length HuJAM1.

“Conservative amino acid substitutions” are those substitutions that, when made, least interfere with the properties of the original protein, i.e., the structure and especially the function of the protein is conserved and not significantly changed by such substitutions. Table 1 below shows preferred conservative amino acid substitutions for an original amino acid in a protein with the most preferred substitution in bold type.

TABLE 1

| Original Residue | Conservative Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| Gly (G) | Ala, Pro |
| His (H) | Arg, Asn, Gln, Lys |
| Ile (I) | Leu, Val, Met, Ala, Phe, |
| | norleucine |
| Leu (L) | Ile, norleucine, Val |
| | Met, Ala, Phe |
| Lys (K) | Arg, Gln, Asn |
| Met (M) | Leu, Ile, Phe |
| Phe (F) | Leu, Val, Ile, Ala, Try |
| Pro (P) | Ala |
| Ser (S) | Thr |
| Thr (T) | Ser |
| Trp (W) | Tyr, Phe |
| Tyr (Y) | Phe, Trp, Thr, Ser |
| Val (V) | Leu, Ile, norleucine, Ala |
| | Phe, Met |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

In the present invention, an “LP polynucleotide” or a “polynucleotide encoding an LP polypeptide” refers to a nucleic acid molecule with a nucleotide sequence of an identified SEQ ID number and its complementary sequence or “complement”. It further includes those polynucleotides of about equal length as the molecule identified by the SEQ ID Number (the “reference molecule”) and capable of hybridizing, under stringent hybridization and wash conditions, to polynucleotide sequences comprising the sequence represented by the SEQ ID Number or the complement thereof. In reference to a polynucleotide, the term “about equal length” means the same number of total nucleotides as the reference molecule plus or minus up to 10, 9, 8, 7, 6, 5, 4, 3, 2, or 1 nucleotides, most preferably plus or minus 0 nucleotides. The addition or deletion of nucleotides may occur anywhere along the length of the polynucleotide molecule and need not be contiguous although the addition or deletion of nucleotides preferably occurs at the 5′ and/or 3′ end(s) when compared to the reference molecule.

A polynucleotide or nucleic acid of the present invention can be composed of any polyribonucleotide or polydeoxyribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded regions. A polynucleotide may contain one or more modified nucleotides. “Modified” bases include, for example, tritylated bases and unusual bases such as inosine. A variety of such modifications can be made; thus, “polynucleotide” embraces chemically, enzymatically, or metabolically modified forms.

“LP variant” means an “active” polypeptide as defined below, having at least about 90% amino acid sequence identity to a reference LP polypeptide. Polypeptide variants include, for instance, variations of LP121A, LP121B, LP121C and LP10034, wherein one or more amino acid residues are added, substituted or deleted, at the N- or C-terminus or within the sequences LP121A, LP121B, LP121C and LP10034 (SEQ ID NOS: 8, 11, 12, and 14), not necessarily contiguously. For example, LP10034 could be the reference polypeptide and the polypeptide altered from the LP10034 polypeptide would be the LP polypeptide variant. Ordinarily, an LP polypeptide variant will have at least about 90% amino acid sequence identity, preferably at least about 91%, 92%, 93%, 94%, 95%, 96%, 97% sequence identity, more preferably at least about 98% sequence identity, even more preferably at least about 99% amino acid sequence identity with the amino acid sequence described (i.e., the reference LP polypeptide), with or without the signal peptide.

“Percent (%) amino acid sequence identity” with respect to the LP amino acid sequences identified herein is defined as the percentage of amino acid residues in a candidate sequence that are identical with the amino acid residues in a reference LP polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as ALIGN, ALIGN-2, Megalign (DNASTAR) or BLAST (e.g., Blast, Blast-2, WU-Blast-2) software. Those skilled in the art can determine appropriate parameters for measuring alignment, including any algorithms needed to achieve maximal alignment over the full length of the sequences being compared. For example, the percent identity values used herein can be generated using WU-BLAST-2 [Altschul et al., Methods in Enzymology 266: 460-480 (1996)]. Most of the WU-BLAST-2 search parameters are set to the default values. Those not set to default values, (i.e., the adjustable parameters) are set with the following values: overlap span=1; overlap fraction=0.125; word threshold (T)=11; and scoring matrix=BLOSUM 62. For purposes herein, a percent amino acid sequence identity value is determined by dividing (a) the number of matching identical amino acid residues between the amino acid sequence of the LP polypeptide of interest and the comparison amino acid sequence of interest (i.e., the sequence against which the LP polypeptide of interest is being compared) as determined by WU-BLAST-2, by (b) the total number of amino acid residues of the LP polypeptide of interest, respectively.

A “LP variant polynucleotide” or “LP variant nucleic acid sequence” means a nucleic acid molecule encoding an active LP polypeptide as defined below having at least 75% nucleic acid sequence identity with an LP polynucleotide identified by a SEQ ID NO. of the present invention. Ordinarily, an LP polypeptide will have at least 75% nucleic acid sequence identity, more preferably at least 80%, 81%, 82%, 83%, 84%, 85%, 86%, 87%, 88%, 89%, even more preferably at least 90%, 91%, 92%, 93%, 94%, 95%, 96% or 97% nucleic acid sequence identity, yet more preferably at least 98% nucleic acid sequence identity, even more preferably at least 99% nucleic acid sequence identity with the nucleic acid sequence of its corresponding nucleic acid represented by a SEQ ID NO. for the reference LP polynucleotide.

“Percent (%) nucleic acid sequence identity” with respect to the LP polynucleotide sequences identified herein is defined as the percentage of nucleotides in a candidate sequence that are identical with the nucleotides in the reference LP sequence after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as ALIGN, Align-2, Megalign (DNASTAR), or BLAST (e.g., Blast, Blast-2) software. Those skilled in the art can determine appropriate parameters for measuring alignment, including any algorithms needed to achieve maximal alignment over the full length of the sequences being compared. For example, percent nucleic acid identity values can be generated using the WU-BLAST-2 (BlastN module) program (Altschul et al., Methods in Enzymology 266: 460-480 (1996)). Most of the WU-BLAST-2 search parameters are set to the default values. Those not set default values (i.e., the adjustable parameters), are set with the following values: overlap span=1; overlap fraction=0.125; word threshold (T)=11; and scoring matrix=BLOSUM62. For purposes herein, a percent nucleic acid sequence identity value is determined by dividing (a) the number of matching identical nucleotides between the nucleic acid sequence of the polypeptide-encoding nucleic acid molecule of interest and the comparison nucleic acid molecule of interest (i.e., the sequence against which the polypeptide-encoding nucleic acid molecule of interest is being compared) as determined by WU-BLAST-2, by (b) the total number of nucleotides of the polypeptide-encoding nucleic acid molecule of interest.

In other embodiments, the LP variant polypeptides are encoded by nucleic acid molecules that encode an active LP polypeptide and which are capable of hybridizing, preferably under stringent hybridization and wash conditions, to nucleotide sequences encoding the full-length LP polypeptide of interest. This scope of variant polynucleotides specifically excludes those sequences that are known as of the filing and/or priority dates of the present application.

The term “mature protein” or “mature polypeptide” as used herein refers to the form(s) of the protein as would be produced by expression in a mammalian cell. For example, it is generally hypothesized that once export of a growing protein chain across the rough endoplasmic reticulum has been initiated, proteins secreted by mammalian cells have a signal peptide (SP) sequence which is cleaved from the complete polypeptide to produce a “mature” form of the protein. Oftentimes, cleavage of a secreted protein is not uniform and may result in more than one species of mature protein. The cleavage site of a secreted protein is determined by the primary amino acid sequence of the complete protein and generally cannot be predicted with complete accuracy. Methods for predicting whether a protein has an SP sequence, as well as the cleavage point for that sequence, are known in the art. A cleavage point may exist within the N-terminal domain between amino acid 10 and amino acid 35. More specifically the cleavage point is likely to exist after amino acid 15 but before amino acid 31. As one of ordinary skill would appreciate, however, cleavage sites sometimes vary from organism to organism and may even vary from molecule to molecule within a cell and cannot be predicted with absolute certainty. Optimally, cleavage sites for a secreted protein are determined experimentally by amino-terminal sequencing of the one or more species of mature proteins found within a purified preparation of the protein.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

The term “amino acid” is used herein in its broadest sense, and includes naturally occurring amino acids as well as non-naturally occurring amino acids, including amino acid analogs and derivatives. The latter includes molecules containing an amino acid moiety. One skilled in the art will recognize, in view of this broad definition, that reference herein to an amino acid includes naturally occurring proteogenic L-amino acids; D-amino acids; chemically modified amino acids, such as amino acid analogs and derivatives; naturally occurring non-proteogenic amino acids such as norleucine, β-alanine, ornithine, etc.; and chemically synthesized compounds having properties known in the art to be characteristic of amino acids. As used herein, the term “proteogenic” indicates that the amino acid can be incorporated into a peptide, polypeptide, or protein in a cell through a metabolic pathway.

The terms “treating”, “treatment” and “therapy” as used herein refer to curative therapy, prophylactic therapy, and preventive therapy. An example of “preventive therapy” is the prevention or lessened targeted pathological condition or disorder. Those in need of treatment include those already with the disorder as well as those prone to have the disorder or those in whom the disorder is to be prevented.

The term “agonist” as used herein refers to a molecule which intensifies or mimics the biological activity of huJAM. Agonists may include proteins, nucleic acids, carbohydrates, small molecules, or any other compound or composition which modulates the activity of huJAM either by directly interacting with huJAM or by acting on component(s) of the biological pathway in which huJAM participates.

The term “antagonist” refers to a molecule which inhibits or attenuates the biological activity of huJAM. Antagonists may include proteins, antibodies, nucleic acids, carbohydrates, small molecules, or any other compound or composition which modulates the activity of huJAM either by directly interacting with huJAM or by acting on component(s) of the biological pathway in which huJAM participates.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption but, rather, is cyclic in nature.

Administration “in combination with” one or more further therapeutic agents includes simultaneous (concurrent) and consecutive administration in any order.

A “therapeutically-effective amount” is the minimal amount of active agent (e.g., an LP polypeptide) which is necessary to impart therapeutic benefit to a mammal. For example, a “therapeutically-effective amount” to a mammal is such an amount which induces, ameliorates or otherwise causes an improvement in the pathological symptoms, disease progression, physiological conditions associated with or resistance to succumbing to the aforedescribed disorder.

“Carriers” as used herein include pharmaceutically-acceptable carriers, excipients, or stabilizers which are nontoxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecule weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol, and PLURONIC™.

“Active” or “activity” in the context of variants of the LP polypeptide refers to retention of biologic function of the unmodified (or wild type) LP polypeptide and/or the ability to bind to a receptor or ligand much as would an unmodified LP polypeptide of the invention, and/or the ability to induce production of an antibody against an antigenic epitope possessed by the LP polypeptide at levels near that of the unmodified LP polypeptide. More specifically, “biological activity” refers to a biological function (either inhibitory or stimulatory) caused by a reference LP polypeptide. Exemplary biological activities include, but are not limited to, the ability of such molecules to induce or inhibit infiltration of inflammatory cells (e.g., leukocytes) into a tissue, to induce or inhibit adherence of a leukocyte to an endothelial or epithelial cell, to stimulate or inhibit T-cell proliferation or activation, to stimulate or inhibit cytokine release by cells or to increase or decrease vascular permeability.

Compositions and Methods of the Invention

The present invention is based in part upon the discovery and synthesis of extracellular huJAM LP proteins (LP121A (SEQ ID NO:11), LP121B (SEQ ID NO:12), LP121C (SEQ ID NO:8), LP10034 (SEQ ID NO:14)) and their use in treating, preventing, and diagnosing cancer, cardiovascular disorders, and immune system disorders such as autoimmune diseases and inflammatory disorders.

1. Preparation of Extracellular HuJAM LP Polypeptides

Nucleic acid encoding an LP polypeptide may be obtained from a cDNA library prepared from tissue believed to possess the LP mRNA and to express it at a detectable level. Libraries can be screened with probes (such as antibodies to an LP polypeptide or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual, Cold Spring Harbor Laboratory Press, NY (1989). An alternative means to isolate the gene encoding LP polypeptide is to use PCR methodology (Sambrook et al., supra; Dieffenbach et al., PCR Primer: A Laboratory Manual, Cold Spring Harbor Laboratory Press, NY (1995)). Further details of the cloning and expression of LP121A and LP10034 are in the Examples herein.

The LP polypeptides of the present invention are extracellular human junction adhesion molecules. These molecules lack the transmembrane domain and cytoplasmic domain that are present in the full-length, membrane-bound human junction adhesion molecules. The LP polypeptides may have a signal peptide sequence to enable protein transport within the cell; however, this signal peptide sequence is not present in the mature huJAM polypeptides as they exist when outside the cell.

A signal peptide, comprised of about 10-30 hydrophobic amino acids, targets the nascent protein from the ribosome to the endoplasmic reticulum (ER). Once localized to the ER, the proteins can be further directed to the Golgi apparatus within the cell. The Golgi distributes proteins to vesicles, lysosomes, the cell membrane, and other organelles. Proteins targeted to the ER by a signal sequence can be released from the cell into the extracellular space. This is the case for the extracellular huJAM polypeptides of the present invention. For example, vesicles containing proteins to be moved outside the cell can fuse with the cell membrane and release their contents into the extracellular space via a process called exocytosis. Exocytosis can occur constitutively or after receipt of a triggering signal. In the latter case, the proteins are stored in secretory vesicles until exocytosis is triggered. Proteins that transit through this pathway are either released into the extracellular space or retained in the plasma membrane. Protein that are retained in the plasma membrane (e.g., full-length huJAM), contain one or more transmembrane domains, each comprised of about 20 hydrophobic amino acid residues. The LP polypeptides of the present invention lack both the transmembrane domain and the downstream cytoplasmic domain of the full-length huJAM.

The common structure of signal peptides from various proteins is typically described as a positively charged n-region, followed by a hydrophobic h-region and a neutral but polar c-region. The (−3, −1) rule states that the residues at positions −3 and −1 (relative to the signal peptide cleavage site) must be small and neutral for cleavage to occur correctly.

In many instances the amino acids comprising the signal peptide are cleaved off the protein during transport or once its final destination has been reached. Specialized enzymes, termed signal peptidases, are responsible for the removal of the signal peptide sequences from proteins. These enzymes are activated once the signal peptide has directed the protein to the desired location.

LP polypeptides of interest may be produced recombinantly, not only directly, but also as a fusion polypeptide with a heterologous polypeptide, which optionally may be a signal sequence or other polypeptide having a specific cleavage site at the N-terminus of the mature protein or polypeptide. In general, the signal sequence may be a component of an expression vector, or it may be a part of the LP polypeptide-encoding DNA that is inserted into such a vector. For E. coli expression, the signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179), or the signal described in WO 90/13646. In mammalian cell expression, mammalian signal sequences may be used to direct secretion of the protein, such as signal sequences from secreted polypeptides of the same or related species as well as viral secretory leaders.

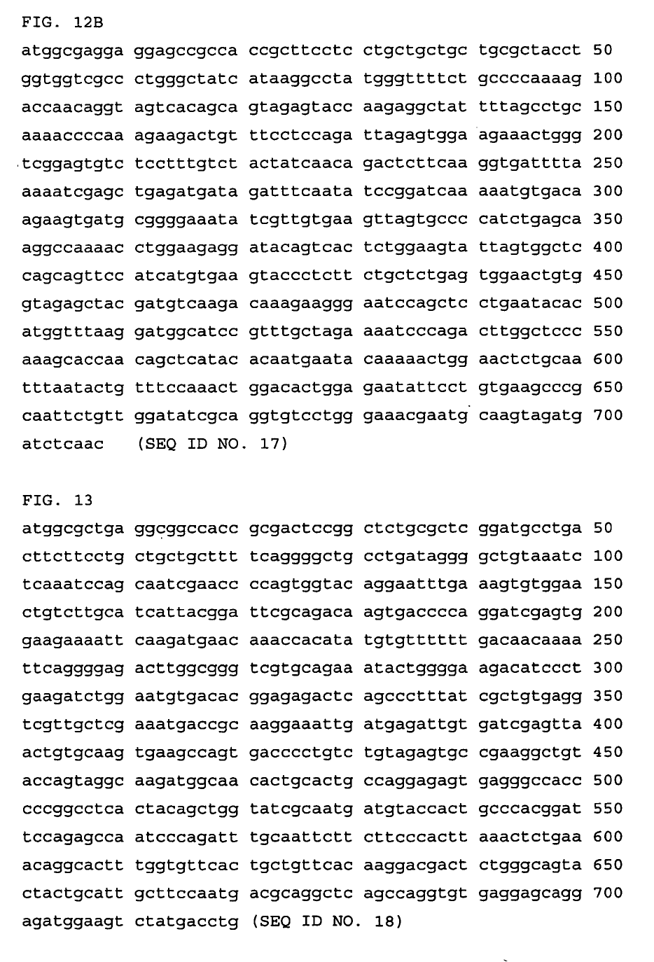

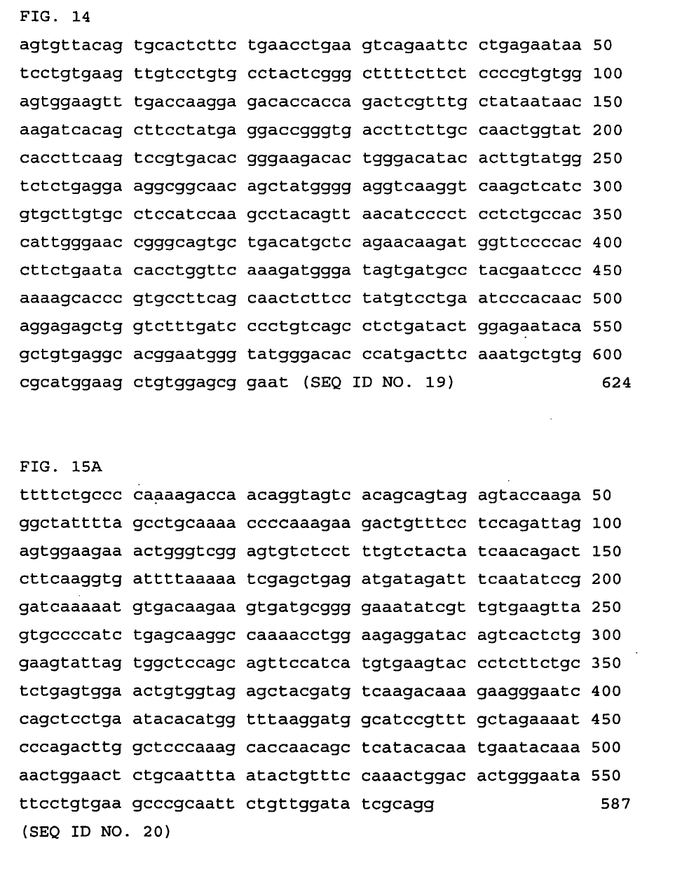

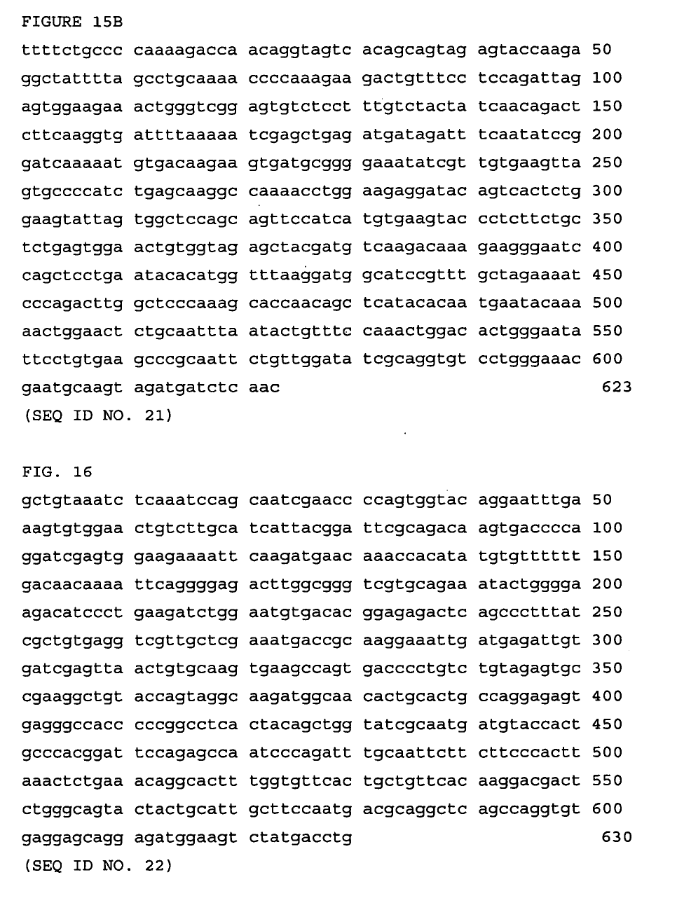

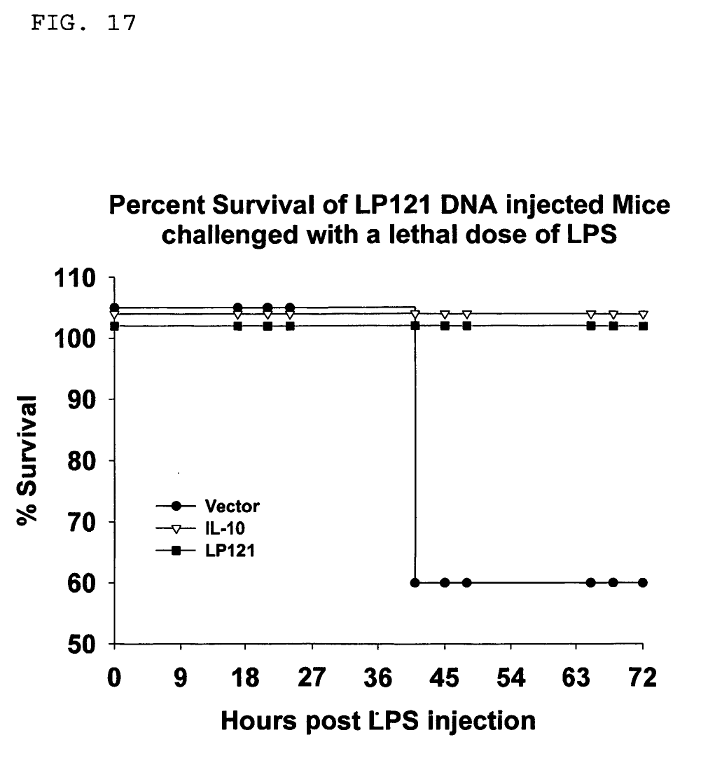

Also provided by the present invention are nucleic acid compositions encoding the LP polypeptides. These nucleic acids are shown in SEQ ID NOS: 15 through 22 (FIGS. 11 through 16 respectively). Also provided are nucleic acids that are homologous or substantially identical or stringently hybridize to the nucleic acid shown in FIG. 11 through 16 as described herein.

2. Extracellular HuJAM LP Polypeptide Variants

The present invention encompasses variants of a polynucleotide sequence encoding an extracellular huJAM polypeptide disclosed in SEQ ID NOS: 15 through 22 and their complementary strands. The present invention also encompasses variants of the extracellular huJAM polypeptide sequences disclosed in SEQ ID NOS: 7 through 14. The term “Variant” refers to a polynucleotide or polypeptide differing from an LP polynucleotide sequence or an LP polypeptide of the present invention, but retaining essential properties thereof. Generally, variants are closely similar overall in structural and/or sequence identity, and, in many regions, identical to an LP polynucleotide or polypeptide of the present invention. The term “variant” is further described in the definitions herein.

The present invention encompasses nucleic acid molecules that comprise as their sole source of extracellular huJAM sequence, or alternatively consist of, a polynucleotide sequence that is at least: 90%, 91%, 92%, 93%, 94% or more preferably at least 95%, 96%, 97%, 98%, or most preferably at least 99% identical to a polynucleotide coding sequence of SEQ ID NOS: 15 through 22 (or a strand complementary thereto); or a polynucleotide sequence encoding a polypeptide of SEQ ID NO: 7 through 14.

Polynucleotides, that encode an extracellular huJAM and stably hybridize to a nucleic acid molecule that comprises as its sole source of extracellular huJAM sequence, or alternatively consists of, a polynucleotide sequence that is at least: 90%, 91%, 92%, 93%, 94% or more preferably at least 95%, 96%, 97%, 98%, or most preferably at least 99% identical to a polynucleotide coding sequence of SEQ ID NOS: 15 through 22 (or a strand complementary thereto); or a polynucleotide sequence encoding a polypeptide of SEQ ID NOs: 7 through 14, under stringent hybridization and wash conditions, are also encompassed by the invention, as are the extracellular huJAM polypeptides encoded by these polynucleotides.

The present invention is also directed to polypeptides that comprise as their sole source of huJAM, or alternatively consist of, an amino acid sequence that is at least: 90%, 95%, 96%, 97%, 98%, 99% identical to a polypeptide sequence of SEQ ID NOs: 7 through 14. A polynucleotide sequence having at least some “percentage identity,” (e.g., 95%) to another polynucleotide sequence, means that the sequence being compared (e.g., the test sequence or candidate sequence) may vary from another sequence (e.g. the reference sequence) by a certain number of nucleotide differences (e.g., a test sequence with 95% sequence identity to a reference sequence can have, on average, up to five point mutations per each 100 contiguous nucleotides of the referent sequence). In other words, for a test sequence to exhibit at least 95% identity to a reference sequence, up to 5% of the nucleotides in the reference may differ, e.g., be deleted or substituted with another nucleotide, or a number of nucleotides (up to 5% of the total number of nucleotides in the reference sequence) may be inserted into the reference sequence. As a practical matter, determining if a particular nucleic acid molecule or polynucleotide sequence exhibits at least about: 90%, 91%, 92%, 93%, 94%, 95%, 96%, 97%, 98% or 99% identity to an LP polynucleotide sequence can be accomplished using known computer programs.

Typically, in such a sequence comparison, one sequence acts as a reference sequence, to which test sequences are compared. When using a sequence comparison algorithm, test and reference sequences are input into a computer, subsequent coordinates are designated, if necessary, and sequence algorithm program parameters are designated. The sequence comparison algorithm then calculates the percentage sequence identity for a test sequence(s) relative to the reference sequence, based on the parameters of a designated program.

Optimal alignment of sequences for comparison can be conducted as described in the definitions herein or, e.g., by the local homology algorithm of Smith and Waterman (1981) Adv. Appl. Math. 2: 482, by the homology alignment algorithm of Needlman and Wunsch (1970) J. Mol. Biol. 48: 443, by the search for similarity method of Pearson and Lipman (1988) Proc. Nat'l Acad. Sci. USA 85: 2444, by computerized implementations of these algorithms (GAP, BESTFIT, FASTA, and TFASTA in the Wisconsin Genetics Software Package, Genetics Computer Group, Madison, Wis.), or by visual inspection (see generally, Ausubel, et al. supra).

A typical method for determining, a best overall match (also referred to as a global sequence alignment) between a test and a referent sequence can be determined using, e.g., the FASTDB computer program based on the algorithm of Brutlag, et al. (1990) Comp. App. Biosci. 6: 237-245. In a FASTDB sequence alignment, the test and reference sequences are, e.g., both DNA sequences. An RNA sequence can be compared by converting U's to T's. The result of a global sequence alignment is given in terms of a percentage identity.

Typical parameters used in a FASTDB alignment of DNA sequences to calculate percent identity are Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500, or the length of the referent nucleotide sequence, whichever is shorter. If the reference sequence is shorter than the test sequence because of 5′ or 3′ terminal deletions, a manual correction must be made to the results. This is because the FASTDB program does not account for 5′ and 3′ terminal truncations of the subject sequence when calculating percent identity. For reference sequences truncated at the 5′ or 3′ terminal ends, relative to the test sequence, the percentage identity is corrected by calculating the number of bases of the test sequence that are 5′ and 3′ of the subject sequence, which are not matched/aligned, as a percentage of the total bases of the test sequence. Whether a nucleotide is matched/aligned is determined by results of the FASTDB sequence alignment. This percentage is then subtracted from the percentage identity, calculated by the above FASTDB program using the specified parameters, to arrive at a final percentage identity score. The corrected score is what is used for the purposes of sequence identity for the present invention. Ordinarily, bases outside the 5′ and 3′ bases of the subject sequence, as displayed by the FASTDB alignment, which are not matched/aligned with the test sequence, are calculated for the purposes of manually adjusting the percent identity score.

For example, a 90 base reference sequence is aligned to a 100 base test sequence to determine percentage identity. The deletions occur at the 5′ end of the reference sequence and therefore, the FASTDB alignment does not show a matched/alignment of the first 10 bases at the 5′ end. The 10 unpaired bases represent 10% of the sequence (number of bases at the 5′ and 3′ ends not matched/total number of bases in the test sequence) so 10% is subtracted from the percentage identity score calculated by the FASTDB program. If the remaining 90 bases were perfectly matched the final percentage identity would be 90%.

In another example, a 90 base reference sequence is compared with a 100 base test sequence. This time the deletions are internal deletions so that there are no bases on the 5′ or 3′ of the subject sequence, which are not matched/aligned with the test. In this case, the percentage identity calculated by FASTDB is not manually corrected. Again, only bases 5′ and 3′ of the subject sequence that are not matched/aligned with the test sequence are manually corrected for. No other manual corrections are made for the purposes of the present invention.

Especially preferred are polynucleotide variants containing alterations, which produce silent substitutions (i.e., no change in amino acid encoded thereby), additions, or deletions, but do not alter the properties or activities of the encoded polypeptide. Nucleotide variants produced by silent substitutions due to the degeneracy of the genetic code are preferred.

A further indication that two nucleic acid sequences of polypeptides are substantially identical is that the polypeptide encoded by the first nucleic acid is immunologically cross reactive with the polypeptide encoded by the second nucleic acid, as described herein. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

A polypeptide exhibiting or having at least about, e.g., 95% “sequence identity” to another amino acid sequence may include, e.g., up to five amino acid alterations per each 100 amino acid (on average) stretch of the test amino acid sequence. In other words, a first amino acid sequence that is at least 95% identical to a second amino acid sequence, can have up to 5% of its total number of amino acid residues different from the second sequence, e.g., by insertion, deletion, or substitution of an amino acid residue.

Alterations in amino residues of a polypeptide sequence may occur, e.g., at the amino or carboxy terminal positions or anywhere between these terminal positions, interspersed either individually among residues in the sequence or in one or more contiguous sections, portions, or fragments within the sequence.

As a practical matter, whether any particular polypeptide sequence exhibits at least about: 90%, 91%, 92%, 93%, 94% 95%, 96%, 97%, 98%, or 99% similarity to another sequence, (e.g., SEQ ID Nos: 7 through 14) can be determined conventionally by using known methods in the art, e.g., a computer algorithm such as ClustalW.

A preferred method for determining the best overall match (also called a global sequence alignment) between two sequences (either nucleotide or amino acid sequences) uses the FASTDB algorithm of Brutlag, et al. (1990) Comp. App. Biosci. 6: 237-245. The result of such a global sequence alignment is given as a percentage of sequence identity, e.g., with 100% representing complete sequence identity.

Typical FASTDB parameters for amino acid alignments are, e.g.,: Matrix=PAM 0, k-tuple=2, Mismatch Penalty=1, Joining Penalty=20, Randomization Group Length=0, Cutoff Score=1, Window Size=sequence length, Gap Penalty=5 Gap Size Penalty=0.05, Window Size=500 or the length of the subject amino acid sequence, whichever is shorter.

If the subject sequence is shorter than the test sequence due to N- or C-terminal deletions, not because of internal deletions, a manual correction must be made to the results. This is because the FASTDB program does not account for N-and C-terminal truncations of the subject sequence when calculating global percent identity. For subject sequences truncated at the N- and C-termini, relative to the test sequence, the percent identity is corrected by calculating the number of residues of the test sequence that are N- and C-terminal of the subject sequence, which are not matched/aligned with a corresponding subject residue, as a percent of the total bases of the test sequence. Whether a residue is matched/aligned is determined by results of the FASTDB sequence alignment. This percentage is then subtracted from the percent identity, calculated by the above FASTDB program using the specified parameters, to arrive at a final percentage identity score. This final percentage identity score is what is used for the purposes of the present invention.

Only residues to the N- and C-termini of the subject sequence, which are not matched/aligned with the test sequence, are considered for the purposes of manually adjusting the percent identity score. That is, only test residue positions outside the farthest N- and C-terminal residues of the subject sequence. For example, a 90 amino acid residue subject sequence is aligned with a 100-residue test sequence to determine percent identity. The deletion occurs at the N-terminus of the subject sequence and therefore, the FASTDB alignment does not show a matching/alignment of the first 10 residues at the N-terminus. The unpaired residues represent 10% of the sequence (number of residues at the N- and C-termini not matched/total number of residues in the test sequence) so 10% is subtracted from the percent identity score calculated by the FASTDB program. If the remaining 90 residues were perfectly matched the final percent identity would be 90%.

In another example, a 90-residue subject sequence is compared with a 100-residue test sequence. This time the deletions are internal deletions so there are no residues at the N- or C-termini of the subject sequence, which are not matched/aligned with the test. In this case, the percent identity calculated by FASTDB is not manually corrected. Once again, only residue positions outside the N- and C-terminal ends of the subject sequence, as displayed in the FASTDB alignment, which are not matched/aligned with the test sequence are manually corrected for. No other manual corrections are to made for the purposes of the present invention.

Variants encompassed by the present invention may contain alterations in the coding regions, non-coding regions, or both. Moreover, variants in which 1-2, 1-5, or 5-10 amino acids are substituted, deleted, or added in any combination are preferred.

Variants may be produced by mutagenesis techniques or by direct synthesis using known methods of protein engineering and recombinant DNA technology. Such variants may be generated to improve or alter the characteristics of an LP polypeptide or may occur unintentionally. For instance, one or more amino acids can often be deleted from the N-terminus or C-terminus of a secreted polypeptide without a substantial loss of biological function.

Moreover, ample evidence demonstrates that polypeptide or polynucleotide variants can retain a biological activity similar to that of the naturally occurring protein. Moreover, even if deleting one or more amino acids from the N-terminus or C-terminus of the polypeptide results in modification or loss of one or more biological functions, other biological activities may be retained.

The invention also encompasses LP polypeptide variants that show a biological activity of the reference huJAM such as, e.g., ligand binding or antigenicity. Such variants include, e.g., deletions, insertions, inversions, repeats, and substitutions selected so as to have little effect on activity using general rules known in the art. For example, teachings on making phenotypically silent amino acid substitutions are provided, e.g., by Bowie, et al. (1990) Science 247: 1306-1310.

One technique compares amino acid sequences in different species to identify the positions of conserved amino acid residues since changes in an amino acid at these positions are more likely to affect a protein function. In contrast, the positions of residues where substitutions are more frequent generally indicates that amino acid residues at these positions are less critical for a protein function. Thus positions tolerating amino acid substitutions typically may be modified while still maintaining a biological activity of a protein.

Another technique uses genetic engineering to introduce amino acid changes at specific positions of a polypeptide to identify regions critical for a protein function. For example, site directed mutagenesis or alanine-scanning mutagenesis (the introduction of single alanine mutations at every residue in the molecule) can be used. (Cunningham and Wells (1989) Science 244: 1081-1085) A resulting mutant can subsequently be tested for a biological activity.

These two techniques have revealed that proteins are surprisingly tolerant of amino acid substitutions and they generally indicate which amino acid changes are likely to be permissive at certain amino acid positions in a protein. For example, typically, most buried amino acid residues (those within the tertiary structure of the protein) require nonpolar side chains, whereas few features of surface side chains are generally conserved. Preferred conservative amino acid substitutions are listed in Table 1.

Besides using conservative amino acid substitutions, other variants of the present invention include, but are not restricted to (i) substitutions with one or more of the non-conserved amino acid residues or (ii) substitution with one or more amino acid residues having a substituent group, or (iii) fusion of the mature polypeptide with another compound, such as a compound to increase the stability and/or solubility of the polypeptide (e.g., polyethylene glycol), or (iv) fusion of the polypeptide with additional amino acids, such as, e.g., an IgG Fc fusion region peptide, or leader or secretory sequence, or a sequence facilitating purification. All such variants would be within the scope of those skilled in the art of molecular biology.

Polypeptide variants containing amino acid substitutions of charged amino acids with other charged or neutral amino acids may produce polypeptides with improved characteristics e.g., such as less aggregation. Aggregation of pharmaceutical formulations both reduces activity and increases clearance due to the aggregate's immunogenic activity (Cleland, et al. (1993) Crit. Rev. Therapeutic Drug Carrier Systems 10: 307-377).

A further embodiment of the invention encompasses a protein that comprises an amino acid sequence of the present invention, as the sole source of huJAM, that contains at least one amino acid substitution, but not more than 20 amino acid substitutions, preferably not more than 15 amino acid substitutions.

Of course, in order of ever-increasing preference, it is highly preferable for an LP polypeptide of the invention to have an amino acid sequence that comprises an amino acid sequence of the present invention as the sole source of huJAM, which contains zero or one, but not more than: 10, 9, 8, 7, 6, 5, 4, 3, 2, or 1 amino acid substitutions; wherein conservative amino acid substitutions are more preferable than non-conservative substitutions.

3. Modifications of Extracellular HuJAM LP Polypeptides

LP polypeptides of the invention are composed of amino acids joined to each other by peptide bonds or modified peptide bonds, i.e., peptide isosteres, and may contain amino acids other than the gene-encoded amino acids. The LP polypeptides may be modified by either natural processes, such as posttranslational processing, or by chemical modification techniques which are well known in the art. Such modifications are well-described in the art. Modifications can occur anywhere in the LP polypeptides, including the peptide backbone, the amino acid side-chains and the amino or carboxyl termini. The same type of modification may be present in the same or varying degrees at several sites in a given LP polypeptide. Also, a given LP polypeptide may contain many types of modifications. LP polypeptides may be branched, for example, as a result of ubiquitination, and they may be cyclic, with or without branching. Cyclic, branched, and branched cyclic LP polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. See, for instance, Creighton, Proteins—Structure and Molecular Properties, 2nd Ed., W.H. Freeman and Company, New York (1993); Johnson, Posttransational Covalent Modification of Proteins, Academic Press, New York, pp. 1-12 (1983); Seifter et al., Meth. Enzymol. 182: 626-46 (1990); Rattan et al., Ann. NY Acad. Sci. 663: 48-62 (1992).

A type of covalent modification of the LP polypeptides included within the scope of this invention comprises altering the native glycosylation pattern of the polypeptide. “Altering the native glycosylation pattern” is intended for purposes herein to mean deleting one or more carbohydrate moieties found in native sequence LP polypeptide and/or adding one or more glycosylation sites that are not present in the native sequences of LP. Additionally, the phrase includes qualitative changes in the glycosylation of the native proteins, involving a change in the nature and proportions of the various carbohydrate moieties present.

Addition of glycosylation sites to LP polypeptides may be accomplished by altering the amino acid sequence thereof. The alteration may be made, for example, by the addition of, or substitution by, one or more serine or threonine residues to the native sequences of LP (for O-linked glycosylation sites). The LP amino acid sequences may optionally be altered through changes at the DNA level, particularly by mutating the DNA encoding the LP polypeptides at preselected bases such that codons are generated that will translate into the desired amino acids.

Another means of increasing the number of carbohydrate moieties on the LP polypeptides is by chemical or enzymatic coupling of glycosides to the polypeptide. Such methods are described in the art, e.g., in WO 87/05330, and in Aplin and Wriston, CRC Crit. Rev. Biochem., pp. 259-306 (1981).

Removal of carbohydrate moieties present on the LP polypeptide may be accomplished chemically or enzymatically or by mutational substitution of codons encoding for amino acid residues that serve as targets for glycosylation. Chemical deglycosylation techniques are known in the art. Enzymatic cleavage of carbohydrate moieties on polypeptides can be achieved by the use of a variety of endo- and exo-glycosidases as described by Thotakura et al., Meth. Enzymol. 138: 350-9 (1987).

Another type of covalent modification of LP polypeptides comprises linking any one of the LP polypeptides to one of a variety of nonproteinaceous 20 polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

LP polypeptides of the present invention may also be modified to form fusion molecules comprising an LP polypeptide as the sole source of extracellular huJAM fused to a heterologous polypeptide. In one embodiment, such a fusion molecule comprises a fusion of an LP polypeptide with a tag polypeptide which provides an epitope to which an anti-tag antibody can selectively bind. The epitope tag is generally placed at the amino- or carboxyl-terminus of LP polypeptide. The presence of such epitope-tagged forms of LP can be detected using an antibody against the tag polypeptide. Also, provision of the epitope tag enables an LP polypeptide to be readily purified by affinity purification using an anti-tag antibody or another type of affinity matrix that binds to the epitope tag.

In an alternative embodiment, the fusion molecule may comprise a fusion of an LP polypeptide which is the sole source of huJAM with an immunoglobulin or a particular region of an immunoglobulin. For a bivalent form of the fusion molecule, such a fusion could be to the Fc region of an IgG molecule.

In yet a further embodiment, the LP polypeptides of the present invention may also be modified in a way to form a fusion molecule comprising an LP polypeptide of the invention which is the sole source of huJAM fused to a leucine zipper at either the N- or C-terminal end of the LP polypeptide. Various leucine zipper polypeptides have been described in the art. It is believed that use of a leucine zipper fused to an LP polypeptide may be desirable to assist in dimerizing or trimerizing soluble LP polypeptides in solution.

4. Expression of LP Polypeptides

a. Expression Vector Construction

Recombinant expression vectors are typically self-replicating DNA or RNA constructs containing a desired gene to be expressed operably linked to a promoter and optionally other control elements recognized in a suitable host cell. The specific type of control elements necessary to effect expression depends on the host cell used and the level of expression desired. Proteins can be expressed in mammalian cells, yeast, bacteria, or other cells under the control of appropriate promoters.

Vectors, as used herein, encompass plasmids, viruses, bacteriophage, integratable DNA fragments, and other vehicles that enable the integration of DNA fragments into the genome of the host although, optionally, expression can occur transiently without integration. Plasmids are the most commonly used form of vector, but many other forms of vectors that perform an equivalent function are also suitable for use (see, e.g., Pouwels, et al. (1985 and Supplements) Cloning Vectors: A Laboratory Manual Elsevier, N.Y.; and Rodriquez, et al. (eds.) (1988) Vectors: A Survey of Molecular Cloning Vectors and Their Uses Buttersworth, Boston, Mass.).

Both expression vectors and cloning vectors contain a nucleic acid sequence that enables the vector to replicate in one or more selected host cells. Such sequences are well known for a variety of bacteria, yeast, and viruses.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement autotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the LP polypeptide-encodingnucleic acid, such as DHFR or thymidine kinase. An appropriate host cell when wild-type DHFR is employed is the CHO cell line deficient in DHFR activity, prepared and propagated as described Urlaub and Chasin, Proc. Natl. Acad. Sci. USA, 77: 4216-20 (1980). A suitable selection gene for use in yeast is the trpl gene present in the yeast plasmid YRp7 [Stinchcomb et al., Nature 282: 39-43 (1979); Kingsman et al., Gene 7: 141-52 (1979); Tschumper et al., Gene 10: 157-66 (1980)]. The trpl gene provides a selection marker for a mutant strain of yeast lacking the ability to grow in tryptophan, for example, ATCC No. 44076 or PEPC1 [Jones, Genetics 85: 23-33 (1977)].

Expression vectors contain a promoter operably linked to the LP polypeptide-encoding nucleic acid sequence to direct mRNA synthesis. Promoters recognized by a variety of potential host cells are well known. Promoters for use in bacterial systems also will contain a Shine-Dalgarno (S.D.) sequence operably linked to the DNA encoding an LP polypeptide of interest.

Transcription of a DNA encoding an LP polypeptide by higher eukaryotes may be increased by inserting an enhancer sequence into the vector. Enhancers are cis-acting elements of DNA, usually about from 10 to 300 bp, that act on a promoter to increase its transcription level. Many enhancer sequences are now known from mammalian genes (globin, elastase, albumin, a-ketoprotein, and insulin). Typically, however, one will use an enhancer from a eukaryotic-cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the LP polypeptide coding sequence.

Expression vectors used in eukaryotic host cells will also contain sequences necessary for the termination of transcription and optionally for stabilizing the mRNA. Such sequences are commonly available from the 5′ and occasionally 3′ untranslated regions of eukaryotic or viral DNAs or cDNAs. These regions contain nucleotide segments transcribed as polyadenylated fragments in the untranslated portion of the mRNA encoding an LP polypeptide.

b. Expression in Host Cells

The description below relates primarily to production of LP polypeptide by culturing cells transformed or transfected with a vector containing LP polypeptide-encoding nucleic acid. It is, of course, contemplated that alternative methods, which are well known in the art, may be employed to prepare LP polypeptides. For instance, the LP sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc. 85: 2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of a LP polypeptide may be chemically synthesized separately and combined using chemical or enzymatic methods to produce a full-length LP.

Host cells are transfected or transformed with expression vectors or cloning vectors described herein for LP polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991) and Sambrook et al., supra. Methods of transfection are known to the ordinarily skilled artisan, for example, CaPO₄ and electroporation.

Suitable host cells for cloning or expressing the nucleic acid (e.g., DNA) in the vectors herein include prokaryote, yeast, or higher eukaryote cells. Suitable prokaryotes include but are not limited to E. coli K12 strain MM294 (ATCC 3 1.446); E. coli Xl 776 (ATCC 3 1.537); E. coli strain W3110 (ATCC 27.325) and KS 772 (ATCC 53.635). Other suitable prokaryotic host cells include Enterobacter, Erwinia, Klebisella, Proteus, Salmonella, Serratia, and Shigeila, as well as Bacilli, Pseudomona, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. Alternatively, in vivo methods of cloning, e.g., PCR or other nucleic acid polymerase reactions, are suitable.

In addition to prokaryotes, eukaryotic microbes such as filamentous fungi or yeast are suitable cloning or expression hosts for LP polypeptide expressing vectors. Saccharomyces cerevisiae is a commonly used lower eukaryotic host microorganism. Many others are used by those in the art.

Suitable host cells for the expression of glycosylated LP polypeptides of the invention are derived from multicellular organisms. Examples of invertebrate cells include insect cells such as Drosophila S2 and Spodoptera Sp, Spodoptera highs as well as plant cells. Examples of useful mammalian host cell lines include Chinese hamster ovary (CHO) and COS cells. Additional examples include the monkey kidney CVl line transformed by SV40 (COS-7, ATCC CRL 1651); human embryonic kidney line [293 or 293 cells subcloned for growth in suspension culture, Graham et al., J. Gen Virol., 36: 59-74 (1977)]; Chinese hamster ovary cells/-DHFR [CHO, Urlaub and Chasin, Proc. Natl. Acad. Sci. USA, 77: 4216-20 (1980)]; mouse sertoli cells [TM4, Mather, Biol. Reprod. 23: 243-52 (1980)]; human lung cells (W138. ATCC CCL 75); human liver cells (Hep G2, HB 8065); and mouse mammary tumor (MMT 060562, ATCC CCL51). The selection of the appropriate host cell is within the skill in the art.

c. LP Polypeptide Purification

Gene amplification and/or expression may be measured in a sample directly, for example, by conventional Southern blotting, Northern blotting to quantitate the transcription of mRNA [Thomas, Proc. Natl. Acad. Sci. USA 77: 5201-5 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal and may be prepared in any mammal. Conveniently, the antibodies may be prepared against a native sequence provided herein or against exogenous sequence fused to an LP polypeptide-encoding DNA and encoding a specific antibody epitope.

Forms of LP polypeptides may be recovered from culture medium or from host cell lysates. The LP polypeptides of the present invention are not membrane-bound. Cells employed in expression of LP polypeptides can be disrupted by various physical or chemical means, such as freeze-thaw cycling, sonication, mechanical disruption, or cell lysing agents.

It may be desirable to purify LP polypeptides away from another recombinant cell polypeptide. The following procedures are exemplary of suitable purification procedures: fractionation on an ion-exchange column; ethanol precipitation; reversed-phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of LP polypeptides. Various methods of protein purification may be employed and such methods are known in the art and described, for example, in Deutscher, Methods in Enzymology 182: 83-9 (1990) and Scopes, Protein Purification: Principles and Practice, Springer-Verlag, NY (1982). The purification step(s) selected will depend, for example, on the nature of the production process used and the particular LP polypeptide produced. Purification of the LP polypeptides of the invention are futher described in the Examples herein.

5. LP Polypeptide Analysis

Many types of analyses can be performed with the LP polypeptides of the present invention to demonstrate their role in the development, pathogenesis, and treatment of cancer, cardiovascular disease and immune related disease, e.g., inflammation. Certain analyses are exemplified in the Examples herein. Protocols for the analyses may be found in Sambrook, et al., supra and other protocol texts used routinely in the art.

The location of tissues expressing the LP polypeptides can be identified by determining mRNA expression in various human tissues. Such a measurement can be made, for example, by Northern blotting, dot blotting, or in situ hybridization based on the sequences provided herein. Alternatively, antibodies may be used that recognize specific duplexes. The location of a gene in a specific tissue can be performed, for example, by Southern blotting.

Cell-based assays using a cell type (optionally known to be involved in a particular disease) are transfected with a vector expressing an LP polypeptide of the invention. Such cells are monitored for phenotypic changes, for example T-cell proliferation by mixed lymphocyte reaction, inflammatory cell infiltration, cytokine levels, JAM expression level variation, ligand binding and reaction to particular antibodies. While transiently-transfected cells can be used, stable cell lines expressing extracellular huJAM are preferred.

Animal models can be used to further understand the role of the LP polypeptides of the invention as demonstrated, for example, in the Examples herein. Example 7 herein demonstrates that the LP polypeptides of the invention may be used for blocking homotypic and/or heterotypic JAM signaling or JAM interactions and therefore can be useful for the prevention, treatment and diagnosis of cardiovascular disease as overexpression of full-length huJAM lead to an enlarged heart phenotype in a Xenopus embryo model. Example 9 herein demonstrates that the LP polypeptides of the invention prevent LPS induced mortality in mice and therefore are useful as an anti-inflammatory agent.

6. Pharmaceutical Compositions

When the coding sequence for an LP polypeptide encodes a protein which binds to another protein as is the case for extracellular huJAM polypeptides of the present invention, the LP polypeptide can be used in assays to identify the other proteins or molecules involved in the binding interaction. By such methods, inhibitors of the receptor/ligand binding interaction can be identified. Proteins involved in such binding interactions can also be used to screen for peptide or small molecule inhibitors or agonists of the binding interaction. Also, the receptor LP polypeptide can be used to isolate correlative ligand(s). Screening assays can be designed to find lead compounds that mimic the biological activity of a native LP polypeptide of the invention or a receptor for an LP polypeptide of the invention. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays, which are well characterized in the art.

The LP polypeptides of the present invention may also be administered via gene therapy protocols. “Gene therapy” includes both conventional gene therapy, where a lasting effect is achieved by a single treatment, and the administration of gene therapeutic agents, which involves the one time or repeated administration of a therapeutically effective DNA or mRNA.

There are a variety of techniques available for introducing nucleic acids into viable cells. The techniques vary depending upon whether the nucleic acid is transferred into cultured cell in vitro or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically, retroviral) vectors and viral coat protein-liposome mediated transfection [Dzau et al., Trends in Biotechnology 11: 205-10 (1993)]. In some situations it is desirable to provide the nucleic acid with an agent that targets the target cells, such as an antibody specific for a cell surface membrane protein or the target cell or a ligand for a receptor on the target cells. Where liposomes are employed, proteins which bind to a cell surface membrane protein associated with endocytosis may by used for targeting and/or to facilitate uptake, e.g., capsid proteins or fragments thereof trophic for a particular cell type, antibodies for proteins which undergo internalization in cycling, proteins that target intracellular localization and enhance intracellular half-life. The technique of receptor-mediated endocytosis is described, for example by Wu et al., J. Biol. Chem. 262: 4429-32 (1987); and Wagner et al., Proc. Natl. Acad. Sci. USA 87: 3410-4 (1990). For a review of gene marking and gene therapy protocols, see Anderson, Science 256: 808-13 (1992).

7. Methods of Treatment

LP121A, LP121B, LP121C, LP10034 and variants thereof are useful for the prevention, diagnosis, and treatment of cancer, cardiovascular disorders and immune system disorders such as autoimmune diseases and inflammatory disorders.

Data presented within the Examples herein demonstrate that overexpression of the full-length, membrane-bound huJAM in the heart leads to an enlarged heart phenotype. Further histological anaysis demonstrated that the heart was dilated. It is contemplated from these data that overexpression of full-length huJAM is associated with conditions of human heart disease and that blocking JAM signaling and/or interactions can be useful for preventing, reversing or improving upon conditions of cardiovascular disease. One method of blocking JAM signaling and homotypic and/or heterotypic interactions would be by overexpression of extracellular huJAM(s).

Further data presented in the Examples herein demonstrate that injection of LPS into mice induces a major inflammatory reaction that, with the dose used, is lethal. Substances that have the ability to reverse this effect have anti-inflammatory properties. DNA encoding extracellular huJAM, when injected into the mice, prevented the LPS-induced mortality demonstrating that huJAM acts as an anti-inflammatory agent.

Particular cancers suitable for treatment with the LP polypeptides of the invention include, but are not limited to, acute myelogenous leukemias including acute monocytic leukemia, acute myeloblastic leukemia, acute promyelocytic leukemia, acute myelomonocytic leukemia, acute erythroleukemia, acute megakaryocyticleukemia, and acute undifferentiated leukemia, etc.; and chronic myelogenous leukemias including chronic myelomonocytic leukemia and chronic granulocyticleukemia. Additional cancers suitable for treatment with the LP polypeptides of the invention inculde, but are not limited to, adenocarcinoma, lymphoma, melanoma, myeloma, Hamartoma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus.

Particular cardiovascular disorder suitable for treatment with the LP polypeptides of the invention include, but are not limited to, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, complications of cardiac transplantation, arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery.

Particular immune system disorders suitable for treatment with the LP polypeptides of the invention include, but are not limited to, inflammatory disorders, acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma.

Therapeutic formulations are prepared for storage by mixing the active ingredient having the desired degree of purity with optional pharmaceutically acceptable carriers, excipients or stabilizers [Remington's Pharmaceutical Sciences 16th edition (1980)], in the form of lyophilized formulations or aqueous solutions.

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition (1980).

The formulations to be used for in vivo administration must be sterile. This is readily accomplished by filtration through sterile filtration membranes.

Therapeutic compositions herein generally are placed into a container having a sterile access port, for example, and intravenous solution bag or vial having a stopper pierceable by a hypodermic injection needle.

Sustained-release preparations may be prepared. Suitable examples of sustained-release preparations include semipermeable matrices of solid hydrophobic polymers containing the therapeutic agent(s), which matrices are in the form of shaped articles, e.g., films, or microcapsules. Examples of sustained-release matrices include polyesters, hydrogels [for example, poly(2-hydroxyethyl-methacrylate), or poly(vinylalcohol)], polylactides, copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. Microencapsulation of recombinant proteins for sustained release has been successfully performed with human growth hormone (rhGH), interferon, and interleukin-2. Johnson et al., Nat. Med. 2: 795-9 (1996); Yasuda et al., Biomed. Ther. 27: 1221-3 (1993); Hora et al., BioTechnology 8: 755-8 (1990); Cleland, “Design and Production of Single Immunization Vaccines Using Polylactide Polyglycolide Microsphere Systems” in Vaccine Design: The Subunit and Adjuvant Approach, Powell and Newman, Eds., Plenum Press, NY, 1995, pp. 439-462 WO 97/03692; WO 96/40072; WO 96/07399; and U.S. Pat. No. 5,654,010.

The sustained-release formulations of these proteins may be developed using polylactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. See Lewis, “Controlled release of bioactive agents from lactide/glycolide polymer” in Biodegradable Polymers as Drug Delivery Systems (Marcel Dekker; New York, 1990), M. Chasin and R. Langer (Eds.) pp. 1-41.

While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity.

The active agents of the present invention are administered to a mammal, preferably a human, in accord with known methods, such as intravenous administration as a bolus or by continuous infusion over a period of time, by intramuscular, intraperitoneal, intracerebral, intracerobrospinal, subcutaneous, intra-articular, intrasynovial, intrathecal, intraoccular, intralesional, oral, topical, inhalation, pulmonary, and/or through sustained release.

Other therapeutic regimens may be combined with the administration of an LP polypeptide of the invention.

For the prevention or treatment of disease, the appropriate dosage of an active agent will depend on the type of disease to be treated, as defined above, the severity and course of the disease, whether the agent is administered for preventive or therapeutic purposes, previous therapy, the patient's clinical history and response to the agent, and the discretion of the attending physician. The agent is suitably administered to the patient at one time or over a series of treatments.

Dosages and desired drug concentration of pharmaceutical compositions of the present invention may vary depending on the particular use envisioned. The determination of the appropriate dosage or route of administration is well within the skill of an ordinary artisan. Animal experiments provide reliable guidance for the determination of effective does for human therapy. Interspecies scaling of effective doses can be performed following the principles laid down by Mordenti and Chappell, “The Use of Interspecies Scaling in Toxicokinetics,” in Toxicokinetics and New Drug Development, Yacobi et al., Eds., Pergamon Press, NY 1989, pp. 4246.

When in vivo administration of an LP polypeptide of the invention is employed, normal dosage amounts may vary from about 1 ng/kg up to 100 mg/kg of mammal body weight or more per day, preferably about 1 pg/kg/day up to 100 mg/kg of mammal body weight or more per day, depending upon the route of administration. Guidance as to particular dosages and methods of delivery is provided in the literature; see, for example, U.S. Pat. Nos. 4,657,760, 5,206,344 or 5,225,212. It is within the scope of the invention that different formulations will be effective for different treatment compounds and different disorders, that administration targeting one organ or tissue, for example, may necessitate delivery in a manner different from that to another organ or tissue. Moreover, dosages may be administered by one or more separate administrations or by continuous infusion. For repeated administrations over several days or longer, depending on the condition, the treatment is sustained until a desired suppression of disease symptoms occurs. However, other dosage regimens may be useful. The progress of this therapy is readily monitored by conventional techniques and assays.

8. Article of Manufacture

In another embodiment of the invention, an article of manufacture containing materials useful for the diagnosis or treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for diagnosing or treating the condition and may have a sterile access port (for example, the container may be an intravenous solution bag or a vial having a stopper pierceable by a hypodermic injection needle). The active agent in the composition is typically an LP polypeptide of the invention. The label on, or associated with, the container indicates that the composition is used for diagnosing or treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

Having generally described the invention, the same will be more readily understood by reference to the following examples, which are provided by way of illustration and not by way of limitation.

TABLE 2

| SEQ | | | | |
|---|---|---|---|---|
| ID | LP# | Name | Type | FIG. |
| 1 | | full-length HuJAM1 | amino acid | 2B |
| 2 | | full-length HuJAM2 | amino acid | 3B |
| 3 | | full-length HuJAM3 | amino acid | 4B |
| 4 | | full-length HuJAM1 | nucleotide | 2A |
| 5 | | full-length HuJAM2 | nucleotide | 3A |
| 6 | | full-length HuJAM3 | nucleotide | 4A |
| 7 | | HuJAM1 (aa⁺ 1-235) | amino acid | 5 |
| 8 | LP121C | HuJAM1 (aa 28-235) | amino acid | 6 |
| 9 | | HuJAM2 (aa 1-224) | amino acid | 7a |
| 10 | | HuJAM2 (aa 1-236) | amino acid | 7b |
| 11 | LP121A | HuJAM2 (aa 29-224) | amino acid | 8a |
| 12 | LP121B | HuJAM2 (aa 29-236) | amino acid | 8b |
| 13 | | HuJAM3 (aa 1-240) | amino acid | 9 |
| 14 | LP10034 | HuJAM3 (aa 31-240) | amino acid | 10 |
| 15 | encodes LP121C* | HuJAM1(aa 1-235) | nucleotide | 11 |
| 16 | encodes LP121A* | HuJAM2(aa 1-224) | nucleotide | 12A |
| 17 | encodes LP121B* | HuJAM2(aa 1-236) | nucleotide | 12B |
| 18 | encodes LP10034* | HuJAM3(aa 1-240) | nucleotide | 13 |
| 19 | encodes LP121C* | HuJAM1(aa 28-235) | nucleotide | 14 |
| 20 | encodes LP121A* | HuJAM2(aa 29-224) | nucleotide | 15A |
| 21 | encodes LP121B* | HuJAM2(aa 29-236) | nucleotide | 15B |
| 22 | encodes LP10034* | HuJAM3(aa 31-240) | nucleotide | 16 |

⁺aa means amino acid
*SEQ ID NOS 15, 16, 17, and 18 encode the extracellular huJAM and the signal peptide; SEQ ID NOS 19, 20, 21, 22 encode the extracellular huJAM without the signal peptide.

### EXAMPLES

General Methods

Commercially available reagents referred to in the examples are used according to manufacturer's instructions unless otherwise indicated. The source of cells identified in the following examples, and throughout the specification, by ATCC accession numbers is the American Type Culture Collection, Manassas, Va. Unless otherwise noted, the present invention uses standard procedures of recombinant DNA technology such as those described or referenced in Sambrook, et al., (1989) Molecular Cloning, A Laboratory Manual, Cold Spring Harbor Press, NY; Ausubel, et al. (1989 and supplements) Current Protocols in Molecular Biology, Green Publishing Associates and Wiley Interscience, NY; Innis et al., or (1990) PCR Protocols: A Guide to Methods and Applications, Academic Press, Inc., N.Y. Unless otherwise noted, the present invention uses standard procedures of protein purification such as those described or referenced in Methods in Enzymology vol. 182 and other volumes in this series; Coligan, et al. (1995 and supplements) Current Protocols in Protein Science, John Wiley and Sons, New York, N.Y.; A Practical Guide to Protein and Peptide Purification for Microsequencing, Academic Press, San Diego, Calif.; or manufacturer's literature on use of protein purification products, e.g., Pharmacia (Piscataway, N.J.) and Bio-Rad (Richmond, Calif.).

### Example 1

### Cloning of LP121A, LP121B, LP121C and LP10034 into a Mammalian Expression Vector

Flag-HIS (FLIS)-tagged version of human LP121A (SEQ ID NO:11), LP121B (SEQ ID NO:12), LP121C (SEQ ID NO:8) and LP10034 (SEQ ID NO:14) are expressed in mammalian cells (e.g., HEK-293EBNA, Cos-7 (ATCC CRL-1651) or HEK-293T (ATCC) in order to generate enough recombinant protein for study. For example, the human LP121A cDNA (SEQ ID NO. 16) is engineered for expression as follows. The pINCY vector containing an Asc I/EcoR V fragment encoding the full-length human JAM2 is used as a template to PCR amplify the coding region of the cDNA.

The pINCY vector (Incyte Genomics, Palo Alto, Calif.) is a derivative of pSPORT1 vector (Invitrogen, Carlsbad, Calif.) created by removing the EcoRI restriction enzyme site by Klenow-filling and religating; removing the Hind III restriction enzyme site by Klenow filling and then inserting into this site a 10-mer EcoRI linker (5′-CGGAATTCCG-3′).

For LP121A, the PCR oligonucleotide primers are 5′-TTACTAGGCTGGCGCGCCACCATGGCGAGGAGGAGC-3′ (SEQ ID NO: 23) containing Asc I endonuclease restriction site (underlined) and 5′-AGGAAGGAATGCGCAAATTATAAAGGATTTTGTGT-3′ (SEQ ID NO: 24) containing an Fsp I restriction site (underlined) for the forward and reverse strands, respectively. The resultant 702-bp PCR generated fragment is cleaved with Asc I and Fsp I, gel-purified and ligated into the mammalian expression vector pEW1969 (a derivative of pJB02, Eli Lilly) that is digested with Asc I and EcoR V to ultimately create vector pEW1969LP121A. Alternative mamalian expression vectors can be used in place of pEW1969. Such vectors are available from multiple companies including Promega Corp. Madison, Wis. and Clontech, Palo Alto, Calif. This construct is designed to express an extracellular huJAM2 molecule spanning amino acids 1-224 of the full-length huJAM2 (including the NH2-terminal amino acids which constitute the signal peptide) and the FLAG/HIS tag at the COOH-terminus of the protein. The expression is under the control of the CMV promoter. The final construct is also designed to produce RNA that is functional in Xenopus embryos in order to study overexpression phenotypes of the protein in Xenopus embryos. One of skill in the art knows how to adapt this method, or alternative methods cited in references hereinabove, for construction of mammalian expression vectors containing SEQ ID NOS:15 through 22.

As an additional cloning example, the human LP10034 cDNA (SEQ ID NO: 18) is engineered for expression as follows. A pINCY vector containing a fragment encoding the full-length of human LP10034 (Incyte Genomics) is used as a template to PCR amplify, using standard PCR conditions, the coding region of the cDNA. The oligonucleotide primers are (5′gatcggcgcgccagccaccATGGCGCTGAGGCGGCCA-3′ (SEQ ID NO: 25)) containing an Asc I endonuclease restriction site (underlined) and (5′ cgccggtttaaacgcCAGGTCATAGACTTCCAT-3′ (SEQ ID NO: 26)) containing a Pme I restriction site (underlined) for the forward and reverse strands, respectively. The resultant 720-bp PCR generated fragment is cleaved with Asc I and Pme I, gel-purified and ligated into a mammalian expression vector, e.g., pPR1 (a derivative of pJB02, Eli Lilly) that is digested with Asc I and Pme I to ultimately create vector pPR1LP10034. This construct is designed to express a truncated molecule (including the NH2-terminal amino acids, which constitute the signal peptide) that is extracellular targeted and the tag at the COOH-terminus of the protein (total of 259 amino acid residues). The expression is under the control of the CMV promoter.

HT-FlagHIS Protein Production

The vectors are separately transiently transfected into mammalian cells (e.g., HEK-293, COS-7 or HEK-293T (Pear, W. S. et al. (1993) PNAS 90: 8392-8396) using FUGENE as described by the vendor (Roche). The recombinant proteins are measured in supernatants collected from the transfected cells by Western-blot using anti-Flag antibody and Coomassie-stained analyses.

### Example 2

### Expression and Purification of LP Polypeptides in E. coli

The bacterial expression vector pQE60 is used for bacterial expression in this example although other bacterial expression vectors are commercially available (QIAGEN, Inc., Chatsworth, Calif.). pQE60 encodes an ampicillin antibiotic resistance gene (Amp^{r}) and contains a bacterial origin of replication (ori), an IPTG-inducible promoter, a ribosome binding site (RBS), six codons encoding histidine (His6 tag) residues that allow affinity purification using nickel-nitrilo-tri-acetic acid (Ni-NTA) affinity resin sold by QIAGEN, Inc., and single restriction enzyme cleavage sites suitable for cloning (i.e., in a multiple cloning site). These elements are arranged such that a DNA fragment encoding a polypeptide of interest can be operably linked in such a way as to produce that polypeptide with the six His residues covalently linked to the carboxyl terminus of that polypeptide. However, a polypeptide coding sequence can optionally be inserted in such a way that translation of the six His codons is prevented and, therefore, a polypeptide is produced with no 6×His tag.

The nucleic acid sequence encoding the desired portion of LP121A, LP121B, LP121C or LP10034 lacking the hydrophobic leader sequence is amplified from a cDNA clone using PCR oligonucleotide primers, which anneal, one upstream (or 5′) and one downstream (or 3′), to the desired portion of the LP polypeptide. Exemplary primers for LP121A and LP10034 are described in Example 1 supra. Additional nucleotides containing restriction sites to facilitate cloning in the pQE60 vector may be added to the 5′ and 3′ sequences, respectively.

The amplified nucleic acid fragments (e.g., those described in Example 1 hereinabove) and the vector pQE60 are digested with appropriate restriction enzymes and the digested DNAs are then ligated together. Insertion of the LP polypeptide DNA into the restricted pQE60 vector places the LP polypeptide coding region including its associated stop codon downstream from the IPTG-inducible promoter and operably linked in-frame with an initiating AUG codon. The associated stop codon prevents translation of the six histidine codons downstream of the insertion point.

The ligation mixture is transformed into competent E. coli cells using standard procedures. E. coli strain M15/rep4, containing multiple copies of the plasmid pREP4, which expresses the lac repressor and confers kanamycin resistance (Kan^{r}), is used in carrying out the illustrative example described herein. This strain, which is only one of many that are suitable for expressing polypeptides, is available commercially from QIAGEN, Inc. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Bacteria containing the desired cloned constructs are grown overnight (O/N) in liquid culture in LB media (Sigma Corp. St. Louis, Mo.) supplemented with both ampicillin (100 μg/ml) and kanamycin (25 μg/ml). The O/N culture is used to inoculate a large culture, at a dilution of approximately 0.1:25 to 1:250. The cells are grown to an optical density at 600 nm (OD600) of between 0.4 and 0.6. Isopropyl-b-D-thiogalactopyranoside (IPTG) is then added to a final concentration of 1 mM to induce transcription from the lac repressor sensitive promoter, by inactivating the lacI repressor. Cells subsequently are incubated further for 3 to 4 hours. Cells then are harvested by centrifugation.

The cells are then stirred for 3-4 hours at 4° C. in 6 M guanidine-HCl, pH 8.0. The cell debris is removed by centrifugation, and the supernatant containing the LP polypeptide is dialyzed against 50 mM Na-acetate buffer pH 6.0, supplemented with 200 mM NaCl. Alternatively, a polypeptide can be successfully refolded by dialyzing it against 500 mM NaCl, 20% glycerol, 25 mM Tris/HCl pH 7.4, containing protease inhibitors.

If insoluble protein is generated, the protein is made soluble according to known method steps. After renaturation, the polypeptide is purified by ion exchange, hydrophobic interaction, and size exclusion chromatography. Alternatively, an affinity chromatography step such as an antibody column is used to obtain purified LP polypeptide. The purified polypeptide is stored at 4° C. or frozen at −40° C. to −120° C.

### Example 3

### Cloning and Expression of LP Polypeptides in a Baculovirus Expression System

In this example, the plasmid shuttle vector pA2 GP is used to insert the cloned DNA encoding the LP polypeptide, without the sequence encoding the signal peptide, into a baculovirus to express the LP polypeptide, using a baculovirus leader and standard methods as described in Summers, et al., A Manual of Methods for Baculovirus Vectors and Insect Cell Culture Procedures, Texas Agricultural Experimental Station Bulletin No. 1555 (1987). This exemplary baculovirus expression vector contains the strong polyhedrin promoter of the Autographa californica nuclear polyhedrosis virus (AcMNPV) followed by the secretory signal peptide (leader) of the baculovirus gp67 polypeptide and convenient restriction sites such as BamH I, Xba I, and Asp 718. The polyadenylation site of the simian virus 40 (SV40) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate viable virus that expresses the cloned polynucleotide.

Other baculovirus vectors can be used in place of the vector above, e.g., pAc373, pVL941 and pAcIM1, as one skilled in the art would readily appreciate, as long as the construct provides appropriately located signals for transcription, translation, secretion and the like, including a signal peptide and an operably-linked AUG start codon as required. Such vectors are described, for instance, in Luckow, et al., Virology 170: 31-39.

The cDNA sequence encoding the mature LP polypeptide of interest lacking the AUG initiation codon (e.g., SEQ ID Nos 19, 20, 21, and 22) and the naturally associated nucleotide binding site, is amplified using PCR oligonucleotide primers corresponding to sequences upstream (5′) and downstream (3′) of the polynucleotide encoding the LP polypeptide of interest. Non-limiting examples include 5′ and 3′ primers having nucleotides corresponding to, or complementary to, a portion of the coding sequence of a LP polypeptide, according to method steps known to those of skill in the art.

The amplified fragment is isolated from a 1% agarose gel using a commercially available kit (e.g., GENECLEAN, Qbiogene, Carlsbad, Calif.). The fragment is then digested with the appropriate restriction enzyme and again is purified on a 1% agarose gel. This fragment is designated herein “F1”.

The plasmid is digested with the corresponding restriction enzymes and optionally, can be dephosphorylated using calf intestinal alkaline phosphatase, using routine procedures known in the art. The DNA is then isolated from a 1% agarose gel using a commercially available kit. This vector DNA is designated herein “V1”.

Fragment F1 and the dephosphorylated plasmid V1 are ligated together with T4 DNA ligase. E. coli HB101 or other suitable E. coli hosts (e.g., XL-1 Blue, Stratagene, La Jolla, Calif.) cells are transformed with the ligation mixture and spread on culture plates. Bacteria are identified that contain the plasmid bearing the polynucleotide encoding the LP polypeptide of interest using the PCR method, in which one of the primers is that used to amplify the nucleic acid and a second primer is from well within the vector so that only those bacterial colonies containing the nucleic acid fragment encoding the LP polypeptide of interest will amplify the DNA. The sequence of the cloned fragment is confirmed by DNA sequencing. This plasmid is designated herein as pBacLP.

Five micrograms of the pBacLP plasmid is co-transfected with 1.0 μg of a commercially available linearized baculovirus DNA (BACULOGOLD™ baculovirus DNA, Pharmingen, San Diego, Calif.), using, for example, the lipofection method described by Felgner, et al., Proc. Natl. Acad. Sci. USA 84: 7413-7417 (1987). 1 μg of BACULOGOLD™ virus DNA and 5 μg of the pBacLP plasmid are mixed in a sterile well of a microtiter plate containing 50 μl of serum-free Grace's medium (Invitrogen). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is rocked back and forth to mix the newly added solution. The plate is then incubated for 5 hours at 27° C. After 5 hours the transfection solution is removed from the plate and 1 ml of Grace's insect medium supplemented with 10% fetal calf serum is added. The plate is put back into an incubator and cultivation is continued at 27° C. for four days.

After four days the supernatant is collected and a plaque assay is performed. An agarose gel with “Blue Gal” (Invitrogen) is used to allow easy identification and isolation of gal-expressing clones, which produce blue-stained plaques. (A detailed description of a “plaque assay” of this type can also be found in the user's guide for insect cell culture and baculovirology distributed by Invitrogen). After appropriate incubation to allow for plaque growth, blue stained plaques are picked with a sterile micropipettor tip. The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and stored at 4° C.

To verify the expression of the LP polypeptide of interest, Sf9 cells are grown in Grace's medium supplemented with 10% heat-inactivated FBS. The cells are infected with the recombinant baculovirus expressing the polypeptide of interest at a multiplicity of infection (“MOI”) of about 2. Six hours later the medium is removed and is replaced with SF900 II medium minus methionine and cysteine (Invitrogen). If radiolabeled polypeptides are desired, 42 hours later, 5 mCi of ³⁵S-methionine and 5 mCi ³⁵S-cysteine (Amersham) are added. The cells are further incubated for 16 hours and then they are harvested by centrifugation. The polypeptides in the supernatant as well as the intracellular polypeptides are analyzed by SDS-PAGE followed by autoradiography (if radiolabeled). Microsequencing of the amino acid sequence of the amino terminus of purified polypeptide can be used to determine the amino terminal sequence of the mature polypeptide and thus the cleavage point and length of the secretory signal peptide.

### Example 4

### Cloning and Expression of LP polypeptide in Mammalian Cells

A typical mammalian expression vector contains at least one promoter element (which mediates the initiation of transcription of mRNA), the polypeptide coding sequence, and signals required for the termination of transcription and polyadenylation of the transcript. Additional optional elements include enhancer(s), a Kozak sequence and an intervening sequence (intron) flanked by donor and acceptor sites for RNA splicing. Highly efficient transcription initiation can be achieved with the early and late promoters from SV40, the long terminal repeats (LTRs) from retroviruses (e.g., RSV, HTLV I, HIV) and the early promoter of the cytomegalovirus (CMV). However, cellular promoters can also be used (e.g., the human actin promoter). Suitable expression vectors for use in practicing the present invention include, but are not limited to, pIRES1neo, pRetro-Off, pRetro-On, PLXSN, or PLNCX (Clontech), pcDNA3.1 (+/−), pcDNA/Zeo (+/−) or pcDNA3.1/Hygro (+/−) (Invitrogen), PSVL and pMSG (Pharmacia, Uppsala, Sweden), pRSVcat (ATCC 37152), pSV2dhfr (ATCC 37146) and pBC12MI (ATCC67109). Suitable mammalian host cells include, but are not limited to, human Hela 293 (ATCC CRL-1573), H9 (ATCC HTB-176), Jurkat cells (ATCC CRL-1990), mouse NIH3T3 (ATCC HB11601), C127 cells (ATCC CRL-1804), Cos 1, Cos 7 and CV 1, quail QC1-3 cells, mouse L cells (ATCC CCL-1) and Chinese hamster ovary (CHO) cells (ATCC CCL-61).

Alternatively, the nucleic acid encoding the polypeptide of interest is expressed in stable cell lines that contain the nucleic acid integrated into a host chromosome. The co-transfection of the nucleic acid encoding the polypeptide of interest along with a gene encoding a selectable marker such as DHRF (dihydrofolate reductase), GPT neomycin, or hygromycin allows the identification and isolation of the transfected cells.

The transfected gene can also be amplified to express large amounts of the encoded polypeptide. The DHFR marker is useful to develop cell lines that carry several hundred or even several thousand copies of the gene of interest. Another useful selection marker is the enzyme glutamine synthase (GS) (Murphy, et al., Biochem. J. 227: 277-279 (1991); Bebbington, et al., BioTechnology 10: 169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of polypeptides.

The expression vectors pC1 and pC4 contain the strong LTR promoter of the Rous Sarcoma Virus (Cullen, et al., Molec. Cell. Biol. 5: 438-447 (1985)) plus a fragment of the CMV-enhancer (Boshart, et al., Cell 41: 521-530 (1985)). Multiple cloning sites (e.g., with the restriction enzyme cleavage sites BamH I, Xba I and Asp 718), facilitate the cloning of the gene of interest. The vectors contain, in addition to the 3′ intron, the polyadenylation and termination signal of the rat preproinsulin gene.

### Example 5

### Cloning and Expression of LP Polypeptides in COS Cells

The expression vector containing the nucleic acid encoding the LP polypeptide of interest, is made by cloning a cDNA encoding the LP polypeptide of interest into the expression vector pcDNAI/Amp or pcDNAIII (Invitrogen).

The expression vector pcDNAI/amp contains: (1) an E. coli origin of replication effective for propagation in E. coli and other prokaryotic cells; (2) an ampicillin resistance gene for selection of prokaryotic cells containing plasmid; (3) an SV40 origin of replication for propagation in eukaryotic cells; (4) a CMV promoter, a multiple cloning site polylinker, an SV40 intron; (5) several codons encoding a hemagglutinin fragment (i.e., an “HA” tag to facilitate purification) or HIS tag (see, e.g, Ausubel, supra) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin polypeptide described by Wilson, et al., Cell 37: 767-778 (1984). The fusion of the HA tag to the target polypeptide allows easy detection and recovery of the recombinant polypeptide with an antibody that recognizes the HA epitope. pcDNAIII contains, in addition, the selectable neomycin marker.

A DNA fragment encoding the LP polypeptide of interest is cloned into the polylinker region of the vector so that recombinant polypeptide expression is directed by the CMV promoter to which it is operably linked. The plasmid construction strategy is as follows. The LP cDNA of a clone is amplified using primers that contain convenient restriction sites.

The PCR amplified DNA fragment and the vector, pcDNAI/Amp, are digested with suitable restriction enzyme(s) and then ligated. The ligation mixture is transformed into E. coli (e.g., strain SURE, Stratagene, La Jolla, Calif.), and the transformed culture is plated on ampicillin LB media plates which then are incubated to allow growth of ampicillin resistant colonies. Plasmid DNA is isolated from resistant colonies and examined by restriction analysis or other means for the presence of the LP polypeptide-encoding fragment.

For expression of recombinant LP121A, LP121B, LP121C or LP10034, COS cells are transfected with an expression vector, as described above, using e.g., DEAE-DEXTRAN. Cells are incubated under conditions appropriate for expression of LP121A, LP121B, LP121C or LP10034 by the vector.

Expression of the LP121A-HA, LP121B-HA, LP121C-HA or LP10034-HA fusion polypeptide is detected by radiolabeling and immunoprecipitation, using methods described in, for example Harlow, et al., Antibodies: A Laboratory Manual, 2nd Ed., Cold Spring Harbor Laboratory Press, Cold Spring Harbor, N.Y. (1988). To this end, two days after transfection, the cells are labeled by incubation in medium containing ³⁵S-cysteine for 8 hours. The cells and the media are collected, and the cells are washed and lysed with detergent-containing RIPA buffer: 150 mM NaCl, 1% NP-40, 0.1% SDS (sodium dodecyl sulfate), 0.5% DOC (deoxycholate), 50 mM TRIS, pH 7.5, as described by Wilson, et al. cited above. Proteins are purified from the cell lysate and from the culture media using a HA-specific monoclonal antibody. The purified polypeptides then are analyzed by SDS-PAGE and autoradiography. An expression product of the expected size is seen in the cell lysate, which is not seen in negative controls.

### Example 6

### Cloning and Expression of LP Polypeptide in CHO Cells

The vector pC4 is used for the expression of LP polypeptide of interest in CHO cells. Plasmid pC4 is a derivative of the plasmid pSV2-dhfr (ATCC Accession No. 37146). The plasmid contains the mouse DHFR gene under control of the SV40 early promoter. CHO cells or other cells lacking dihydrofolate activity that are transfected with these plasmids can be selected by growing the cells in a selective medium (alpha minus MEM, Invitrogen) supplemented with methotrexate. The amplification of the DHFR genes in cells resistant to methotrexate (MTX) has been well documented (see, e.g., F. W. Alt, et al., J. Biol. Chem. 253: 1357-1370 (1978); J. L. Hamlin and C. Ma, Biochem. et Biophys. Acta 1097: 107-143 (1990); and M. J. Page and M. A. Sydenham, Biotechnology 9: 64-68 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach can be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains the LTR strong promoter of the Rous Sarcoma Virus (Cullen, et al., Molec. Cell. Biol. 5: 438-447 (1985)) plus a fragment isolated from the enhancer of the immediate early gene of human CMV (Boshart, et al., Cell 41: 521-530 (1985). Downstream of the promoter are BamH I, Xba I, and Asp 718 restriction enzyme cleavage sites that allow insertion of the genes. Downstream of these cloning sites the plasmid contains the 3′ intron and polyadenylation site of the rat preproinsulin gene. Other high efficiency promoters can also be used for expression, (e.g., human b-actin promoter, SV40 early or late promoters, or the LTR from other retroviruses). Clontech's Tet-Off and Tet-On gene expression systems and similar systems can be used to express the LP polypeptide of interest in a regulated way in mammalian cells (M. Gossen, and H. Bujard, Proc. Natl. Acad. Sci. USA 89: 5547-5551 (1992)). For the polyadenylation of the mRNA, polyadenylation signals, (e.g., from the human growth hormone or globin genes) can be used. Stable cell lines carrying a gene of interest integrated into the chromosomes can also be selected upon co-transfection with a gene expressing a selectable marker such as gpt, G418 or hygromycin. It is advantageous to use more than one selectable marker in the beginning, e.g., G418 plus methotrexate.

The plasmid pC4 is digested with appropriate restriction enzyme(s) and then dephosphorylated using calf intestinal phosphatase by procedures known in the art. The vector is then isolated from a 1% agarose gel.

The DNA sequence encoding the LP polypeptide of interst is amplified using PCR oligonucleotide primers corresponding to sequences 5′ and 3′ to the sequence of interest.

The amplified fragment is digested with suitable endonuclease(s) and then purified again on a 1% agarose gel. The isolated fragment and the dephosphorylated vector are then ligated with T4 DNA ligase. E. coli (e.g., HB101 or XL-1 Blue cells) are then transformed and bacteria are identified that contain the fragment inserted into plasmid pC4 using, for instance, using restriction enzyme analysis.

Chinese hamster ovary (CHO) cells lacking an active DHFR gene are used for transfection. Five micrograms of the expression plasmid pC4 is cotransfected with 0.5 μg of the plasmid pSV2-neo using e.g., lipofectin. The plasmid pSV2-neo contains a dominant selectable marker, the neomycin resistance gene from Tn5 encoding an enzyme that confers resistance to a group of antibiotics including G418. The cells are seeded in alpha minus MEM supplemented with 1 μg/ml G418. After 2 days, the cells are trypsinized and seeded in hybridoma cloning plates (Greiner, Germany) in alpha minus MEM supplemented with 10, 25, or 50 ng/ml of methotrexate plus 1 μg/ml G418. After about 10-14 days clonal colonies are independently trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then independently transferred to a new well of a 6-well plate containing even higher concentrations of methotrexate (1 mM, 2 mM, 5 mM, 10 mM, 20 mM). The same procedure is repeated until clones are obtained which grow at a concentration of 100-200 mM methotrexate. Expression of the desired gene product is analyzed, for instance, by SDS-PAGE and Western blot or by reverse phase HPLC analysis.

### Example 7

### Overexpression of huJAM1, huJAM2, huJAM3, LP121A, LP121B, LP121C and LP10034 in Xenopus Embryos

Injecting RNA that encode novel proteins into early Xenopus laevis embryos may result in various phenotypes in the early tadpoles (i.e. split axis, anteriorization etc.) that help elucidate the function of these proteins.

The DNA encoding the LP polypeptide of interest is first cloned into an expression vector (e.g., PR1) containing the T7 RNA polymerase promoter. Plasmid DNA containing the LP cDNA insert is linearized with Not I restriction enzyme (Gibco BRL, #15441-025) and in vitro transcribed using MESSAGE MACHINE (Ambion, #1344) containing T7 RNA polymerase. The RNAs produced are examined on a 1.25% agarose gel to confirm success of the transcription reaction as well as the appropriate size and concentration of the RNA product. The RNA is translated in vitro using the biotin in vitro translation kit (Roche, #1559451) to determine if RNA is of sufficient quality to produce protein of predicted molecular weight. Finally, RNA is diluted to 200 μg/ml with Rnase-free water and stored at −20° C. until ready for microinjection into Xenopus embryos.

Female Xenopus frogs are injected with 300 U/frog of human choronic gonadotropin (Sigma Corp., St. Louis, Mo.) to induce egg laying. Eggs are harvested from the females and combined with macerated testis to fertilize the eggs in vitro.

Fertilized eggs are dejellied with 2% cysteine in water (pH 8.0), rinsed, and transferred to 1×MR (0.1 M NaCl, 1.8 mM KCl, 2.0 mM CaCl₂, 1.0 mM MgCl₂ and 50 mM HEPES-NaOH). Embryos develop at room temperature or 15° C. until signs of the first cleavage furrow appear. Embryos are then transferred to 4% Ficoll (Sigma Corp.) in 1×MR prior to injection.

Five nl (1 ng) of RNA is injected into both cells of a two-cell stage embryo. Uninjected controls are also maintained. Embryos are left in 4% Ficoll/1×MR solution until they reach the blastula stage of development, then they are switched to 0.1×MR with 4% Ficoll and continue to develop at 18° C. Embryos are observed for morphological effects during the next 2-3 days. Phenotypes of tadpoles are recorded by photography using a digital camera and a dissecting microscope.

Histological analysis of embryos is also performed. The tadpoles are fixed in 3% formaldehyde and embedded in paraffin. Tissues are prepared for analysis by removing the parafin with xylene and then gradually rehydrating the tissue with graded solutions of ethanol and water. Sections are then stained with hematoxylin and eosin solutions, coverslipped and viewed under a light microscope. Compared to control embryos of three day old tadpoles, the embryos injected with full-length huJAM2 have enlarged dilated hearts.

Animal Cap Assays with LP Polypeptides

Animal cap explants from early Xenopus embryos normally give rise to cells solely of the ectodermal lineage. Addition or expression of foreign proteins can change the fate of these cells into other lineages (endodermal and mesodermal) thereby helping to functionate newly discovered proteins.

RNA-injected embryos at stage 8 (Niewkoop and Faber, 1967) are harvested for animal cap studies. Vitelline membranes are removed from the embryos and a sheet of cells from the animal pole (animal cap) is isolated. The animal caps are grown in a 96-well tissue culture dish (Costar) with animal cap buffer [0.2×MR, 1% BSA (Sigma Corp.), and 59 μg/ml Gentamicin (Sigma Corp.)] overnight at room temperature until control embryos reach approximately stage 17-19 (Niewkoop and Faber, 1967).

In addition, animal cap assays can also be performed with uninjected embryos with addition of the LP protein (1-20 nM) to the animal cap buffer.

Results of these assays are monitored by morphology, RT-PCR for tissue specific gene expression, and immunohistochemistry for visualization of tissue type and distribution within the explant.

Overexpression of full-length, membrane-bound HuJAM2 (SEQ ID NO: 2) results in embryos with ventral edema and dilated hearts. Overexpression of the truncated, extracellular form of huJAM2 [LP121A (SEQ ID NOs: 11)] or the truncated, extracellular form of huJAM3 [LP10034 (SEQ ID NO:14)} results in no observable altered phenotype in this assay. Since only overexpression of the full-length, membrane-bound HuJAM results in heart edema phenotype in the Xenopus embryo, it is contemplated that this molecule is involved in diseases of the heart in humans. HuJam display homotypic and/or heterotypic binding; e.g., it is known that huJAM2 has both types of binding. Extracellular huJAM, e.g., LP121A, LP121B, LP121C, LP10034, are able to neutralize or block binding of protein molecules to the overexpressed full-length, membrane-bound huJAM in diseased states of the heart. This blocking or neutralization is accomplished by extracellular huJAM binding the extracellular portion of a membrane-bound huJAM and preventing it from interacting with receptors and/or ligands that exist on other cells or that are soluble in the extracellular space. This blocking or neutralization prevents, reverses, and/or treats the diseased heart condition.

### Example 8

### Tissue Distribution of LP mRNA

Non-radioactive northern blot analysis is performed to examine gene expression in human tissues. First, a control probe, Human G3PDH cDNA Control Probe (Clontech #9805-1), is labeled with digoxigenin using DIG-High Prime Labeling kit (Roche #1585606).

The cDNA encoding the LP polypeptide of interest is cloned into a pPR1 or XenoFLIS vector using PCR conditions known to a person skilled in the art. This may be accomplished using the following PCR mixture: 5 μL PCR buffer and 5 μL PCR DIG mix from PCR DIG Probe Synthesis Kit (Roche # 1636090); 0.75 μL Enzyme Mix (Expand High Fidelity, Roche #1732641); 5 μL of 10 μM pPR1 probe primer (5′-TGCAAAGCTTGGCGCGCC-3′ SEQ ID NO: 27); 5 μL of 10 μM FLAG Probe Primer (5′-CTTGTCGTCGTCATCCTTGTAGTCG-3′ SEQ ID NO: 28); 1 μL plasmid DNA (long); and 28.25 μL sterile water. The cDNA is labeled using this mixture and the following PCR method: one cycle of 95° C. for two minutes; thirty to forty cycles of 95° C. for thirty seconds, 60° C. for thirty seconds, and 70° C. for 1.5 minutes/1 kb of inserted cDNA; one cycle of 72° C. for ten minutes; and a 4° C. soak cycle.

Next, the samples are hybridized to human multiple tissue northern (MTN) blot membranes (Clontech #7780-1) human cardiovascular system MTN blot (Clontech #7791-1) and human endothelial cells, diseased, and normal heart using DIG Easy Hyb (Roche # 1603558). Membranes are prehybridized with DIG Easy Hyb for 1 hour at 50° C. The DIG DNA probes prepared above are denatured at 100° C. for 10 minutes then immediately placed on ice, diluted with DIG Easy Hyb (2 μl probe to 10 ml Easy Hyb), and the membranes are incubated overnight at 50° C. with the diluted hybridization/probe mixture.

Detection of the membranes is accomplished using CDP-Star chemiluminescent substrate (Roche # 1685627) and X-ray film. The membranes are washed twice in 2×SSC at 5° C. for 30 minutes each, twice in 0.5×SSC at 50° C. for 30 minutes each and twice in 0.1×SSC at 50° C. for 30 minutes each. The membranes are blocked with DIG Blocking Buffer (Wash and Block Buffer Kit, Roche # 1585762) for one hour to overnight at room temperature, and anti-digoxigenin-AP Fab fragments (Roche # 1093274) are then added at a 1:20,000 dilution for 2 hours to overnight at room temperature. The membranes are washed three times in 1× wash buffer, equilibrated in detection solution (DIG Wash and Block Buffer Kit, Roche # 1585762) then incubated with CDP-Star (Roche # 1685627) at 1:100 dilution for 5 minutes at room temperature. After lightly blotting, the membranes are placed between two pieces of transparency film and exposed to X-ray film for various exposure times to ensure detection of all possible bands.

Northern blot data indicate that the main tissue of LP121A expression is the heart and placenta, with lesser amounts present in brain, smooth muscle, kidney, small intesting, and lung tissues and even less still in the colon, thymus, spleen and liver. All areas of the heart express huJAM2. Fetal heart appears to have a more intense signal compared to adult heart. Thus, the DNA encoding extracellular huJAM and their respective mRNA and proteins are contemplated to be useful in the treatment of cardiovascular diseases. In addition, huJAM2 may be involved in inflammatory diseases of the kidney, small intestine, lung, colon and liver.

Human Endothelial, Diseased, and Normal Heart Northern Blots

Total RNA is isolated using the RNAqueous-Midi Kit (Ambion, #1911) following the manufacturer's protocol. Twenty micrograms of RNA, diluted in 3 volumes of NorthernMax formaldehyde loading dye (Ambion, #8552) is separated with a precast, 1.25% agarose, 1×MOPS RNA gel (BioWhittaker, #52922). Gel is stained for RNA integrity with 0.5 ug/ml of ethidium bromide for 20 min. and destained with distilled water for 10 min. RNA is transferred to a BrightStar-Plus membrane (Ambion, #10102) using NorhternMax Transfer Buffer (Ambion, #8672). RNA is crosslinked and stored at −20° C. until ready to hybridize by methods already described above.

Homotypic Binding Assays

A BIAcore 2000 instrument is used to detect real-time binding between immobilized LP121A, LP121B, LP121C or LP10034 and soluble LP121A, LP121B, LP121C or LP10034. LP121A, LP121B, LP121C or LP10034 is diluted to a concentration of 50 μg/mL in 10 mM sodium acetate buffer at pH 5.0. LP121A, LP121B, LP121C or LP10034 is immobilized to a CM5 sensor chip using the amine coupling method.

LP121A, LP121B, LP121C and LP10034 proteins are diluted in HBS-EP buffer. Samples are injected over LP121A, LP121B, LP121C or LP10034 and control surfaces using the kinject method. Samples containing 5 μg/mL and 1 μg/mL of protein are injected at 30 μL/min for three minutes with a ninety second dissociation time. LP121A, LP121B, LP121C and LP10034 and control surfaces are then regenerated with glycine-hydrochloride at pH 3.0.

### Example 9

### Protein Binding in Human Tissue

Binding of LP121A, LP121B, LP121C and LP10034 proteins to human tissues is determined by protein staining with fluorescent dye. The following human tissues are used in this assay:

TABLE 3

LP polypeptide

| Tissue | LP121A | LP10034 |
|---|---|---|
| Kidney | 0 | 0 |
| Liver | 0 | 0 |
| Heart | 0 | 0 |
| Lung | 0 | 0 |
| Spleen | 0 | 0 |
| Pancreas | 1 | 1 |
| Gut | 1 | 1 |
| thymus | 0 | 0 |
| Ampulla | 0 | 0 |
| Bone marrow | nd | 2 |
| Prostate | 1 | 0 |
| Ovary | 1 | 0 |
| Skin | 0 | 0 |
| Vessels | 0 | 0 |
| Skeletal | | |
| Muscle | 0 | 0 |
| Brain | 0 | 0 |
| Peri nerve | 0 | 0 |
| Breast | 0 | 0 |
| Lymph node | 2 | 0 |
| Adipose | 0 | 0 |
| Adr. Gland | 0 | 0 |

All tissues are fixed with 3% paraformaldehyde and embedded in paraffin. Tissues are prepared for analysis by removing the paraffin with xylene then gradually rehydrating the tissue with graded solutions of ethanol and water. Antigen retrieval is performed to unmask antigenic sites so that antibodies can recognize the antigen. This is accomplished by soaking the tissue in citrate buffer (Dako, Carpinteria, Calif.) for twenty minutes at 80 to 90 degrees C. followed ten minutes at ambient temperature. The tissue is then washed in tris-buffered saline (TBS) containing 0.05% TWEEN®-20 and 0.01% thimerosol. To minimize non-specific background staining, the tissue is soaked in non-serum protein block (Dako) for forty-five minutes, after which the protein block is removed by blowing air over the tissue.

The tissue is exposed for two hours to the FLAG-HIS tagged LP121A, LP121B, LP121C or LP10034 protein at 10 μg/mL. Following exposure, the tissue is washed twice with tris-buffered saline (TBS) containing 0.05% TWEEN®-20 and 0.01% thimerosol. The tissue sample is then incubated for one hour with mouse anti-FLAG antibody at 10 μg/mL. Subsequently, the tissue is washed twice with tris-buffered saline (TBS) containing 0.05% TWEEN®-20 and 0.01% thimerosol. Next, the tissue is exposed to rabbit anti-mouse Ig with Alexa 568, a fluorescent dye, at 10 μg/mL for one hour, followed again by two washes with TBS containing 0.05% TWEEN®-20 and 0.01% thimerosol. Finally, the tissue is coverslipped with fluorescence mounting media, and the fluorescence is measured. A positive fluorescence reading indicates that the protein binds with antigens on the tissue, suggesting that the protein binds to that tissue indicating localization of possible ligand or receptor for that LP polypeptide.

### Example 9

### In Vivo Function of LP121A, LP121B, LP121C or LP10034 with Endotoxin Challenged Mice

Endotoxin is a lipopolysaccharide (LPS) from gram negative bacterial cells which immediately induces systemic release of inflammatory mediators such as TNF and IL-1. Mice injected with LPS usually die within 48 hours of injection. DNA injected into the tail veins of mice will translocate to the liver where protein encoded by the DNA will be produced and secreted into the blood stream. Injection of Il-10 DNA protects mice from death when challenged with LPS. Injection of LP121A, LP121B, LP121C or LP10034 will test their anti-inflammatory properties.

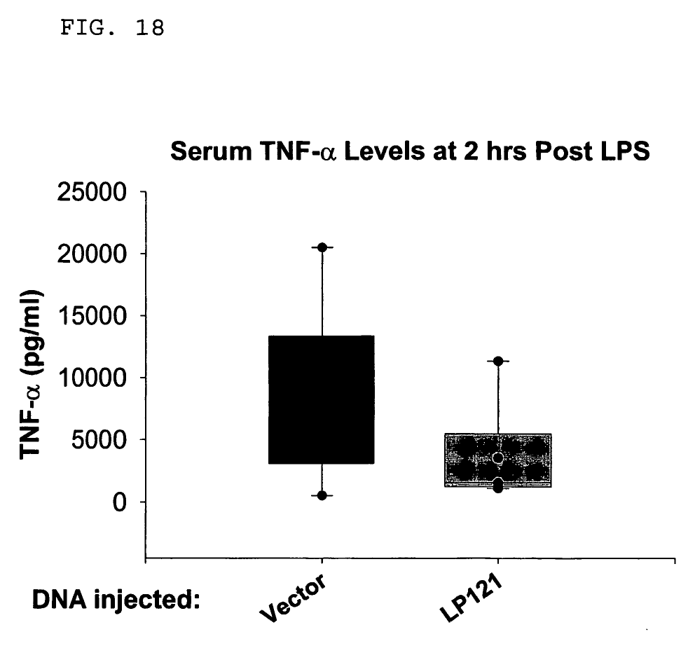

Plasmid DNA for LP121A, LP121B, LP121C or LP10034 under control of the CMV promoter is prepared from DH5-α E. coli cells using an endotoxin free DNA isolation kit (Qiagen #12362). Il-10 is cloned behind a CMV promoter in the pcDNA3.1/v5-His-TOPO vector (Invitrogen #K4800-01) as a positive control DNA (Il-10 abrogates the effects of the endotoxin). DNA is quantified using a spectrophotometer and prepared for injection with the TRANSIT in vivo gene delivery kit (Mirus Corp). Mice (Harlan) aged 6-8 week and weighing on average 24.5 g are injected with 20 ug/mouse (in 2.4 mls) of LP121A, LP121B, LP121C, LP10034, IL-10 or vector alone DNA into the tail vein. Twenty-four hours post-DNA injection mice are challenged with endotoxin and D-galactosamine (10 μg/mouse and 6 mg/mouse respectively). Mice are monitored 3× daily for 72 hours to determine survival (FIG. 17). Two hours post LPS injection, retro-orbital bleeds are done to collect serum for analysis of cytokines by Luminex (BioRad, #171-F12080)(FIG. 18).

It is contemplated that the LP121A DNA injected into the mice is transcribed then translated into protein and secreted into the blood stream. It is further contemplated that the mice injected with LP121A survive the LPS effect by reducing or eliminating the inflammatory response resulting from the LPS. Since vector alone does not protect the mice from the LPS challenge, it is concluded that the LP polypeptide protects the mice from death in this assay. It is further contemplated from this data that full-length, transmembrane huJAM2 is involved in the transmigration of immune cells from the blood stream into the tissues and that extracellular huJAM inhibit this function of full-length huJAM.

In addition, LP121A protects mice from death without altering cytokine secretion in the animals. Alterations in cytokine expression levels can abate inflammatory responses. Based on the hypothesis for the role of LP polypeptides of the invention in inhibiting inflammation, no changes in assayed cytokine levels is consistent with this hypothesis. Mice injected with LP121A (extracellular huJAM2) DNA (SEQ ID NO. 16) are protected from death (5/5 mice survived) when challenged with LPS. The graph in FIG. 17 shows one experiment of two performed in the LPS model. In the second experiment performed, LP121A proteced 80 of the mice (4/5 mice survived), vector alone protected 0% of the mice, and IL-10 protected 100% of the mice 48 hours after LPS injection. LP121A protected the mice from death without increasing cytokine levels (such as TNF-α) in the blood stream.

### Example 10

### In Situ Assays

Human Peripheral Blood T Cell Assay

Tissue culture plates (Nunc) are coated with 0.5 μg/ml or 50 ng/ml anti-human CD3 monoclonal antibody (Pharmingen, #30111A) overnight at 4° C. Next, varying concentrations (1-3 μg/ml) of Lp121A, LP121B, LP121C, LP10034, or CD28 monoclonal antibody (Pharmingen, #33741A) are added for 4 hours. Human blood was diluted 2-fold with RPMI-1640(Gibco BRL, #22400) and is separated over a Ficoll (Sigma, #F8636) gradient by centrifugation at 2000 rpm for 5 min. Cells are collected from the gradient and washed twice in RPMI-1640 and centrifuge at 1000 rpm for 5 min. Cells are resuspeded in 0.5% BSA/PBS. T cells are isolated using the Miltenyi BioTec Pan T cell Isolation kit (530-01). Cells are washed in media, suspended in RPMI-1640 with 10% FCS. Cells are plated at 2×10⁶ cells/ml in 100 ul/well, incubated for 3 days at 37° C. with 5% CO₂. Supernatants are collected to measure cytokine analysis (Il-beta1, Il-2, Il-4, Il-5, Il-10, GMCSF, IFN-gamma, and TNF-alpha) with Luminex kits (BioRad,#171-A12080) following the manufacturer's instructions. Then 100 μl of medium is added back to the cells and cells are pulsed with 1 μCi of ³H-thymidine overnight at 37° C. with 5% CO₂. Next, cells are frozen and thawed for lysis and harvested with a Skatron Semiautomatic cell harvester. Total ³H-thymidine incorporation is measured by a beta-scintillation counter, with mean of triplicate results reported.

Mouse T-cell Assay

Tissue culture plates are coated with 0.5 μg/ml of anti-mouse CD3 monoclonal antibody (Pharmingen, #30111A) in DPBS by adding 100 μl/well overnight at 4° C. Wells are then washed with DPBS and coated with 0.23 μg/ml of anti-CD28 monoclonal antibody. Various concentrations (1-3 μg/ml) of LP121A, LP121B, LP121C or LP10034 are added to the plates for 4 hours at 37° C. Mouse T cells are harvested from lymph nodes and spleens from 4 Balb/c mice. Tissue is ground between 2 frosted glass slides and pipetted through a 70 μM nylon cell strainer into a 50 ml tube. Cells are washed 3× with RPMI-1640 with 10% FCS and centrifuged at 1000 rpm for 5 min. Cells are resuspended in DPBS with 0.5% BSA. Murine T cells are positively selected using CD90 microbeads and autoMACS system (Miltenyi). Cells are resuspended to 2×10⁶ cells/ml in RPMI-1640 with 10% FCS and antibiotic. One hundred microliters of cell suspension is added to each well of a 96-well plate. Cells are incubated for 3 days, then supernatants are collected to measure cytokine release. One hundred microliters of medium is added back to the cells and the cells are pulsed with 1 μCi ³H-thymidine for 8 hours. Next, the cells are frozen and thawed for lysis and harvested with a Skatron Semiautomatic cell harvester. Total ³H-thymidine incorporation is measured by a beta-scintillation counter, with mean of triplicate results reported.

RAW264.7 Bioassay RAW264.7 cells are plated at 1×10⁵ cells/well in a 96 well plate in 200 μl final volume in DMEM (GibcoBRL, #11995) with 10% fetal bovine serum (FBS). Cells are incubated overnight at 37° C. and 5% CO₂, then LP121A, LP121B, LP121C or LP10034 are added at 1 μg/ml for 6 hours at 37° C. and 5% CO₂. Next, the cells are incubated with or without LPS (Sigma, 100 ng/ml) for an additional 24 hours. Cytokines are measured 24 hours after LPS addition using 30 μl of supernatant and the BioPlex Mouse Cytokine Assay (BioRad, #171-F12080). Nitric oxide is measured at 40 hours after LPS addition using 50 μl of supernatant and Griess Reagent (Sigma Corp., #G4410).

A549 Bioassay

S549 cells are plated at 3×10⁴ cells/well in a 96 well plate in 200 μl final volume in HamsF12K (GibcoBRL, #11765-054) with 10% FBS and 0.15% sodium bicarbonate. Cells are incubated 24 hours at 37° C. and 5% CO₂ then starved for 16-18 hours in serum free medium. LP121A, LP121B, LP121C or LP10034 are added to the medium and incubated 6 hours at 37° C. before adding TNF-alpha (R&D Systems, #210-TA-010, 50 ng/ml). Cells are incubated for an additional 24 hours at 37° C., then 10 μl of supernatant is used for cytokine detection using the BioPlex Human Cytokine Assay (BioRad, #171-A12080). Il-8 ELISAs (R&D System, #D8050) use 5 μl of supernatant.

HUVEC Bioassay

HUVEC cells are plated at 4×10⁴ cells/well in a 96 well plate in 200 μl EGM (Clonetics). Cells are incubated for 24 hours at 37° C. then starved for 16-18 hours in serum free medium (EGM with no supplements). LP121A, LP121B, LP121C or LP10034 are added (1 μg/ml) and incubated for 6 hours at 37° C. before adding TNF-alpha (R&D Systems, #210-TA-010, 10 ng/ml). Cells are incubated an additional 24 hours, then cytokines are measured using 15 μl of supernatant and BioPlex Human Cytokine Assay (BioRad,# 171-A12080).

Primary Macrophage Bioassay

Mice are injected with 3 ml of 3% Brewer thioglycollate intraperinoneally 72 hours before harvesting peritoneal macrophages. Mice are sacrificed and 10 ml of DMEM+10% FBS is injected into the peritoneum. The solution is aspirated after gentle washing. The cells are plated at 3×10⁵ cells/well, 200 μl volume in DMEM+10% FBS in a 96 well plate. Cells are incubated for 2 hours at 37° C. then medium is removed and LP121A, LP121B, LP121C or LP10034 is added (diluted in DMEM+10% FBS). Cells are incubated an additional 24 hours at 37° C. then cytokines are measured using 30 μl of supernatant and the Mouse BioPlex Cytokine Assay (BioRad, #171-F12080).

Adhesion Assays

One hundred microliters of anti-FLAG antibody is coated onto a 96 well plate at 5 μg/ml in PBS. Next, 5 μmol of LP121A, LP121B, LP121C, LP10034 (each fused to a FLAG epitope), or irrelevant FLAG-tagged protein is captured by the bound anti-FLAG antibody. Various cell lines and human leukocytes are labeled with calcein (Molecular Probes, Eugene, Oreg.) at 50 μg/ml for 25 min. at 37° C. Cell binding is performed for 90 min. at 37° C. with 2.5×10⁵ cells/well in binding buffer (Tris-buffered saline, 1 mM CaCl₂, 1 mM MgCl₂, and 1 mM MnCl₂). Wells are washed three times with binding buffer and lysed with 50 mM Tris (pH 7.5), 5 mM EDTA, and 1% IGEPAL. Fluorescence is read in a Cytofluor 4000 (Perseptive Biosystems) with excitation at 485/20 nm and emission at 530/25 nm. Specific binding is determined by removal of values obtained by the irrelevant FLAG tagged protein.

### Example 11

### Transendothelial Cell Migration Assay

Isolation of Monocytes and Neutrophils

Whole human blood is collected into a heparinized syringe by a phlebotomist. The blood is then carefully layered over Polymorphprep solution (Nycomed Pharma As) at a ratio of 40 ml blood to 10 ml Polymorphprep in a 50 ml conical centrifuge tube. The tube is then centrifuged at 500×g for 30 minutes in a swinging bucket rotor at 20° C. At the conclusion of the run, two bands of leukocytes are present. The top band contains the mononuclear cells from which the monocytes will be isolated. The lower band contains primarily neutrophils. The top band is carefully aspirated and diluted 10× with RPMI-1640 (Gibco BRL, #22400). The lower band is then aspirated and diluted 10× with RPMI-1640. Both populations are washed twice with RPMI-1640 and placed on ice. The neutrophils are now ready for counting and loading.

Monocytes are further isolated using the AutoMACS sorting system and monocyte isolation kit (Miltenyi Biotec). According to the protocol, 10,000,000 cells are suspended in 60 μl PBS/BSA (0.5%)/EDTA (2 mM). The suspension then receives 20 ul Fc blocking reagent and 20 μl antibody-hapten cocktail. This cocktail labels all cells except monocytes. After a 20 min. 6° C. incubation, the cells are washed one time and resuspended in 60 ul buffer. The suspension then receives 20 ul Fc blocking reagent and 20 μl anti-hapten microbeads. After a 20 min., 6° C. incubation, the cells are washed one time and resuspended in 1.0 ml buffer. The cells are then run through the AutoMACS cell sorter and the negative fraction used as the monocyte population.

Loading of both the neutrophils and monocytes is accomplished using calcein AM from Molecular Probes. Cells are suspended in RPMI-1640. The suspension receives pluronic F-127 and calcein AM at final concentrations of 0.1% and 5 uM. The cells are incubated at room temperature for 30 minutes. After washing twice, the fluorescent cells can be resuspended in the buffer of choice for assay.

Chemotaxis Through Cell Monolayers

Human umbilical vein endothelial cells (HUVECs) are cultured in Transwells (8 μm pore, Costar) and grown to confluence. Soluble LP121A, LP121B, LP121C or LP10034 are added at various concentrations (0.2-5 μg/ml) to HUVEC monolayer, neutrophils, or monocytes for 30 min. at 37° C. Labeled monocytes and neutrophils are diluted to a concentration of 1.8×10⁶ cells/ml and 750 μl of cells are added/well for 60 min. at 37° C. Chemokines MCP-1 and/or MCP-3 (R&D Systems, #279-MC-010 and #282-P3-010) may be added to the lower chamber at 100 ng/ml. The number of labeled cells in the bottom chamber is measured using the Cytofluor 4000 (Perseptive Biosystems).

Liquid Bone Marrow Assay

Human bone marrow cells (Poieteics) are cultured in IMDM+30% FBS in V-bottom polyproylene 96 well plates for 10-11 days in the presence of differing combinations of stem cell factor (R&D systems #255-SC-010, 10 ng/ml), Il-3 (R&D Systems #203-IL-010, 0.1 ng/ml), erythropoietin (R&D Systems #286-EP-250, 1 U/ml), transforming growth factor beta (R&D Systems #240-B-002, 10 ng/ml), macrophage colony stimulating factor (R&D Systems #216-MC-005, 40 ng/ml), and LP121 or LP10034 (400 ng/ml). Feeding occurs on days 4 and 8. Cells are centrifuged in plate and resuspended in 10 μg/ml unconjugated IgG. Suspensions are incubated at 4° C., with both anti CD14-FITC (1:100, Miltenyi Biotech) and anti CD36-PE (1:20, Pharmingen, #30985X) for 20 min. Cells are washed with PBS then transferred to flow cuvettes containing 1 ml PBSA (fv) containing 100 μl Flow Count beads (Coulter, #7547053). Cell analysis is done by flow cytometry.

### Example 12

### Soluble HuJAM2 and HuJAM3 Interactions

Self-association and binding of 2oluble HuJAM3 to 2oluble HuJAM2 were investigated using analytical ultracentrifuge (AUC), size-exclusion chromatography (SE-HPLC), and isothermal titration experiment (ITC), and surface plasma resonance.

Sedimentation equilibrium experiments were performed using an XLA analytical ultracentrifuge. A 110 μl aliquot of sample solution at protein concentration of about 0.2 to 0.4 mg/mL in PBS buffer was added to the sample column of the cell and 120 μl of the PBS buffer was added to the reference column of the cell. The equilibrium run was performed at 20° C. and 16000 rpm overnight until the system reached equilibrium. At equilibrium, the system exhibits a concentration gradient along the column. For an ideal single species, a plot of 1 nC versus r² gives a straight line and the slope is directly related to the molecular weight of macromolecule by the following equation
M(1−νρ)ω²/2RT=dlnC/dr²=slope
Where: M is the molecular weight
ν is the partial specific volume (mL/g)
ρ is the density of the solvent (g/mL)
ω is rotor speed (radians/second)
R is gas constant (8.314*10⁷ erg/mol K)
T is temperature in Kelvin
C is concentration of the solute at radius r

The density of 1.00 g/cm³ for PBS buffer solution was used and partial specific volume was calculated based amino acid compositions (V-bar of 0.712 mL/g and 0.722 mL/g for soluble HuJAM3 and soluble HuJAM2). The apparent molecular weights measured are summarized in the Table 4 below and compared to that measured by MALDI mass spectrometry. The difference between the calculated molecular weight and that determined by MALDI indicates that the protein is glycosylated. Soluble HuJAM2 contains two potential N-linked glycosylation sites and soluble HuJAM3 contains 3 potential N-linked sites. These sites can be fully or partially glycosylated when the protein is expressed from a mammalian cell line. The molecular weight determined by AUC indicates that both soluble HuJAM2 and soluble HuJAM3 are dimers in solution.

TABLE 4
Molecular weight

| | Calculated MW | MW by MALDI | MW by AUC |
|---|---|---|---|
| Sample | (Da) | (Da) | (Da) |
| Human LP121 | 23995 | 28295 | 60000 |
| Human LP10034 | 26135 | 30177 | 58637 |

Size-exclusion chromatography was performed using a TSK-3000 column coupled with an on-line light scattering detector, miniDAWN (Wyatt Technology Corp). Phosphate buffer saline containing 0.5M NaCl buffer at pH 7.4 with 0.0005% sodium azide was used as mobile phase. The flow rate of 0.5 mL/min and run time of 35 min were used. Forty microliters of protein sample (0.2 to 0.6 mg/mL) was injected onto the column. Molecular weight of the peak was determined using the software supplied for the instrument (ASTRA 4.73.04). Soluble HuJAM3 runs as dimer on the size-exclusion column, while soluble HuJAM2 runs as a monomer under these conditions suggesting the dimer form is not very stable and can be dissociated into monomers under the conditions used for SE-HPLC experiment. When two proteins were mixed in 1:1 molar ratio, the monomeric peak for soluble HuJAM2 disappeared, indicating that soluble HuJAM3 and soluble HuJAM2 can form heterodimer.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is to be understood that no limitation with respect to the specific examples presented is intended or should be inferred. The disclosure is intended to cover by the appended claims modifications as fall within the scope of the claims.

1. A purified extracellular huJAM polypeptide comprising at least 95% sequence identity to an amino acid sequence selected from the group consisting of SEQ ID NOS: 8, 11, 12, and 14.

2. The extracellular huJAM polypeptide of claim 1, wherein said polypeptide lacks a transmembrane domain.

3. The extracellular huJAM polypeptide of claim 2, wherein said polypeptide is capable of binding a huJAM ligand.

4. The purified extracellular huJAM polypeptide of claim 2 encoded by a polynucleotide, wherein said polynucleotide lacks sequence encoding a transmembrane domain.

5. A purified extracellular huJAM polypeptide comprising an amino acid sequence selected from the group consisting of SEQ ID NOS: 8, 11, 12, and 14.

6. The extracellular huJAM polypeptide of claim 5, wherein said polypeptide lacks a transmembrane domain.

7. The extracellular huJAM polypeptide of claim 6, wherein said polypeptide is capable of binding a huJAM ligand.

8. The extracellular huJAM polypeptide of claim 6 encoded by a polynucleotide, wherein said polynucleotide lacks sequence encoding a transmembrane domain.

9. An isolated polynucleotide encoding an extracellular huJAM polypeptide and having at least 95% sequence identity to a nucleic acid sequence selected from the group consisting of SEQ ID NOS: 15, 16, 17, 18, 19, 20, 21, and 22.

10. The polynucleotide of claim 9 wherein said polynucleotide lacks sequence encoding a transmembrane domain.

11. An isolated polynucleotide encoding an extracellular huJAM protein wherein said polynucleotide is selected from the group consisting of SEQ ID NOS: 15, 16, 17, 18, 19, 20, 21, and 22.

12. A vector comprising a polynucleotide encoding an extracellular huJam protein as the sole source of huJAM nucleotide sequence.

13. The vector of claim 12, which is an expression vector.

14. A host cell transfected with the vector of claim 12.

15. A host cell transfected with the expression vector of claim 13.

16. The host cell of claim 15, wherein said cell is a CHO cell.

17. The host cell of claim 15, wherein said cell is an E. coli cell.

18. The host cell of claim 15, wherein said cell is an Sf9 cell.

19. The host cell of claim 15, wherein said cell is a yeast cell.

20. A method for producing a polypeptide, the method comprising the steps of:
a) culturing the host cell of claim 15 under conditions suitable for the expression of the polypeptide from the said polynucleotide and
b) recovering the polypeptide from the cell culture medium.

21. An isolated polypeptide produced by the method of claim 20.

22. A chimeric molecule comprising the extracellular huJAM of claim 1 fused to a heterologous amino acid sequence wherein the extracellular huJAM of claim 1 is the sole source of huJAM.

23. The chimeric molecule of claim 22, wherein said heterologous amino acid sequence is an epitope tag sequence.

24. The chimeric molecule of claim 22, wherein said heterologous amino acid sequence is an Fc region of an immunoglobulin.

25. A chimeric molecule comprising the extracellular huJAM of claim 5 fused to a heterologous amino acid sequence wherein the extracellular huJAM of claim 5 is the sole source of huJAM.

26. The chimeric molecule of claim 22, wherein said heterologous amino acid sequence is an epitope tag sequence.

27. The chimeric molecule of claim 22, wherein said heterologous amino acid sequence is an Fc region of an immunoglobulin.

28. A pharmaceutical composition comprising the extracellular huJAM of claim 1 in conjunction with a suitable pharmaceutical carrier.

29. A pharmaceutical composition comprising the extracellular huJAM of claim 5 in conjunction with a suitable pharmaceutical carrier.

30. A method of treating an immune system disorder comprising administering a therapeutically effective amount of the extracellular huJAM of claim 1 to a mammal having said disorder.

31. The method of claim 30, wherein the immune system disorder is an immune deficiency, an autoimmune disease or an inflammatory disorder.

32. A method of treating an immune system disorder comprising administering a therapeutically effective amount of the extracellular huJAM of claim 5 to a mammal having said disorder.

33. The method of claim 32, wherein the immune system disorder is an immune deficiency, an autoimmune disease or an inflammatory disorder.

37. A method of treating cancer comprising administering a therapeutically effective amount of an extracellular huJAM of claim 1 to a mammal having said cancer.

38. A method of treating a cardiovascular disorder comprising administering a therapeutically effective amount of an extracellular huJAM of claim 1 to a mammal having said disorder.

39. A method of treating wound healing comprising administering a therapeutically effective amount of an extracellular huJAM of claim 1 to a mammal in need of such treatment.

40. An article of manufacture comprising a container, label and therapeutically effective amount of an extracellular huJAM of claim 1 in combination with a pharmaceutically effective carrier.
